(19) United States
(12) Patent Application Publication
(10) Pub. No.: US 2009/0199855 A1
(43) Pub. Date: Aug. 13, 2009

(54) SYSTEM AND METHOD FOR CONSERVING OXYGEN DELIVERY WHILE MAINTAINING SATURATION

(76) Inventor: James M. Davenport, Kingman, AZ (US)

Correspondence Address: DAVIS & BUJOLD, P.L.L.C., 112 PLEASANT STREET, CONCORD, NH, 03301, US

(21) Appl. No.: 11/718,352

(22) PCT Filed: Oct. 31, 2005
(86) PCT No.: PCT/US2005/039531

§ 371 Date: Jan. 14, 2009

Related U.S. Application Data
(60) Provisional application No. 60/624,047, filed on Nov. 1, 2004.

(51) Int. Cl. A61M 16/00

(52) U.S. Cl. 128/204.23

(57) ABSTRACT

A system and method, for maintaining a predetermined level of a treatment gas in a patient while conserving use of the treatment gas, comprising a source of the treatment gas, a sensing device for sensing a breathing cycle of a patient, a conserver for controlling intermittent supply of the treatment gas to the patient in response to the sensed breathing cycle. In a first mode, when the sensing device senses breathing, the treatment gas is intermittently supplied to the patient at a supply rate coordinated with the breathing cycle. In a second mode, when the sensing device is unable to sense breathing, the treatment gas is supplied to the patient at a second intermittent cycle, determined independently of the patient breathing cycle, which is selected to overlap an assumed patient breathing cycle such that at least a desired level of the treatment gas is maintained in the patient.

### CROSS REFERENCE TO RELATED APPLICATIONS

The present patent application is related to U.S. Pat. No. 5,697,364 issued on Dec. 16, 1997 to Chua et al. for an INTERMITTENT GAS-INSUFFLATION APPARATUS.

### FIELD OF THE INVENTION

The present invention relates to a system and method for insufflating a quantity of a gaseous treatment fluid, which may be, for example, a gas such as oxygen or some other gas, a mixture of gases, including air enriched with additional oxygen or depleted of nitrogen to increase the proportion of oxygen therein, a gas or gas mixture carrying dispersed liquid droplets or solid particles, into an entrance of a respiratory system of a breathing patient to maintain a desired saturation level of the treatment gas in the patient while conserving consumption of the treatment gas, but maintaining an acceptable level of treatment gas in the patient under fault conditions, such as a loss of inhalation/exhalation data from the patient or a loss of power in the system.

### BACKGROUND OF THE INVENTION

Systems for the controlled delivery of a treatment gas which may be, for example, a gas such as oxygen or some other gas, a mixture of gases, including air enriched with additional oxygen or depleted of nitrogen to increase the proportion of oxygen therein, a gas or gas mixture carrying dispersed liquid droplets or solid particles, are well known in the prior art and typically comprise a gas source, a gas regulator and a gas conserver which cooperate to deliver the treatment gas from the source to the patient in a controlled manner. For example, in a typical treatment gas delivery system 1P, as diagrammatically illustrated in FIG. 1, the treatment gas such as oxygen or any gas or gas/particulate/droplet mixture, is generally contained in and provided from a gas source 2P which may be, for example, a gas tank containing a treatment gas such as gaseous or liquid oxygen or gas concentrator delivering the treatment gas, and is supplied through a supply conduit 4P to a regulating device, which is typically of the type of regulator referred to as a conserver 6P. The conserver 6P will then output a desired quantity of the treatment gas through a patient conduit 8P at a desired pressure and flow rate, typically, at a flow rate of from 1 to 6 liters per minute, for example. In a typical system the treatment gas, such as oxygen, is typically delivered to the patient by a cannula or some other face piece worn by the patient which feeds the oxygen into the nasal cavities or possibly the mouth cavity of the patient. From there, the delivered oxygen is inhaled by the patient into the lungs for absorption into the blood stream of the patient.

As indicated, the conserver 6P typically includes pressure sensing equipment 10P associated with the patient 12P to detect the breathing cycles of the patient. That is, each breathing cycle includes an inhalation interval and an exhalation interval, also respectively referred to as a negative pressure interval and a positive pressure interval, and, in general, the sensing equipment 10P will determine when the patient is commencing an inhalation, or negative pressure, interval and will then supply the treatment gas to the patient for the duration of time during which the patient is inhaling. When the sensing equipment 10P determines that the patient's inhalation interval has ceased, the conserver 6P will discontinue the supply of treatment gas to the patient 12P until the sensing equipment 10P again senses that the patient 12P is commencing another inhalation interval, at which time the supply of treatment gas to the patient 12P is again commenced. By intermittently interrupting the delivery of oxygen to the patient, the quantity of treatment gas delivered to the patient is conserved, e.g., reduced or minimized, without seriously impacting the saturation level of the patient 12P.

It must be noted that the gas source 2P may typically comprise either a tank or some other storage structure, such as for gaseous or liquified oxygen, or of a treatment gas generator, such as a conventional concentrator. In the case of a concentrator, which is typically employed when the treatment gas is oxygen or oxygen enriched air, for example, the concentrator employs molecular sieves to absorb some of the nitrogen from the room air, thereby providing a treatment gas to the conserver 6P having a proportionally increased oxygen content.

There are a number of drawbacks associated with the treatment gas systems of the prior art, one of which is that the systems of the prior art typically dispense the treatment gas, such as oxygen, only when the system senses the beginning of an inhalation interval by a patient. One of the problems associated with this technique is that there is a time delay from the time when an inhalation interval is detected and the conserver is actuated to the time that the oxygen is actually delivered to the patient. As a result of this delay, the initial volume of air inhaled by the patient primarily comprises the partially exhaled breath of the patient as well as the unconcentrated room air which does not contain an elevated or increased amount of oxygen therein. As a result of this, the air initially inhaled by the patient does not have an elevated or increase level of oxygen therein.

This problem is more serious than first appears, however, and the full complexity of the problems associated with the gas dispensing systems of the prior art can be fully understood only when the actual nature of a patient breathing cycle is considered and understood in detail. First considering the breathing cycle itself, a plot of the breathing pressure of the patient as a function of time over the breathing cycle, the pressures occurring in a breathing cycle generally appear as a modified sine wave. That is, during the exhalation interval there is a positive breathing pressure having a form generally corresponding to one half of a sine wave form as the pressure rises then falls relative to ambient air pressure. Correspondingly, during the inhalation interval, there is a negative breathing pressure having a form generally corresponding to the second half of a sine wave form as the pressure continues to fall after termination of the exhalation interval and then rises relative to the ambient air pressure. In actuality, the sine wave form of the breathing cycle is skewed so that the exhalation interval of the skewed sine wave constitutes, on an average, about two thirds of the breathing cycle while the inhalation interval of the skewed sine wave constitutes, on an average, about one third of the breathing cycle.

Furthermore, the respiratory system of the patient includes the passageway to the lungs comprising the nares of the nose, the nasal cavity and the trachea which together provide a conduit for transporting ambient atmospheric air to and fro a person's lungs. This passageway is anatomically dead space that, after the exhalation interval, is now filled with exhaled air which, in turn, becomes the first initial quantity of inhaled air during the subsequent inhalation interval. By way of example only, on the average, this anatomically dead space retains about the first one third (⅓) of the quantity of air for the next inhalation. The remaining two thirds (⅔) of the quantity of air required for breathing is provided by fresh ambient atmospheric air during the subsequent inhalation interval. However, only about one half (½) of this fresh ambient air actually reaches the lungs for gaseous exchange, i.e., only about the second one third (⅓) of the required air (or the first one half (½) of the fresh air) is carried to the lungs while the last one third (⅓) of the required air (or the second one half (½) of the fresh air) never actually reaches the lungs and remains in the anatomically dead space. Therefore, on the average, only 16% to 17% of the breathing cycle brings fresh air or fresh air combined with the insufflation gas to the lungs and this occurs only during the first one half (½) of the inhalation interval of the breathing cycle.

As a consequence of the two factors discussed above, it is apparent that only the gas delivered during a relatively small part of the breathing cycle actually reaches the patient's lungs to be of benefit to the patient, and continuous flow systems are obviously very inefficient in terms of the consumption and use of the treatment gas.

In response to this problem, many systems and devices of the prior art have included oxygen-conserving features, which are generally characterized as either “on demand” systems or “on the go” systems. In general, “on demand” means that oxygen is not delivered to the patient until after the beginning of the inhalation interval of the breathing cycle and that no oxygen is delivered to the patient during any portion of the exhalation interval of the breathing cycle. Since oxygen was not delivered to the patient during the exhalation interval, which constitutes two thirds of the entire breathing cycle, significant quantities of oxygen were conserved.

Examples of “on demand” systems include U.S. Pat. No. 4,462,398 and U.S. Pat. No. 4,519,387 to Durkan et al. wherein a control circuit, responsive to a sensor, operates a valve to supply pulses of respirating gas through a single hose cannula to a respiratory system of a patient when a negative pressure, indicative of the initial stage of inhalation or inspiration, is sensed by the sensor. The pulse of gas delivered to the respiratory system can have a preselected pulse profile. This method provides for supplying a fixed volume of supplemental respiratory gas per unit of time. The volumetric flow rate of the supplemental respiratory gas is preset and the time duration of each application of the supplemental respiratory gas is also preselected, thereby providing a fixed volume of respiratory gas after the beginning of inhalation. Also, this method provides for a minimal delay interval between successive applications of respiratory gas and such delay interval is also predetermined since the time interval for respiratory gas flow is preset for a time less than the time of the inspiration.

Another prior art supplemental oxygen delivery system designed to conserve respiratory gas by delivering oxygen “on demand” only during inhalation is described in U.S. Pat. No. 4,612,928 to Tiep et al. which discloses both a method and apparatus for supplying a gas to a body. The apparatus and method are employed to minimize the amount of oxygen needed to maintain a specific oxygen concentration level in the blood of an individual. The apparatus includes a transducer and other circuit components to obtain a first series of pulses or signals corresponding to the individual's breath rate. A divider or counter processes the signals or pulses of the first series to create a second series of pulses or signals corresponding to periodic pulses or signals of the first series. The pulses or signals of the second series are used to periodically open a valve to deliver oxygen to the individual at about the start of the inhalation interval of the individual's periodic breathing cycles.

In further examples, such as U.S. Pat. Nos. 4,457,303 and 4,484,578, recognize that oxygen delivered at the end of the inhalation interval of the breathing cycle is wasteful. These two patents describe respirator apparatuses and methods therefor. In brief, a fluidically-operated respirator comprises an apneic event circuit and a demand gas circuit. The apneic event circuit comprises a variable capacitance device and an exhaust means which rapidly discharges fluid from the circuit when inhalation occurs. The demand gas circuit of the respirator supplies the respirating gas to a patient at the beginning of inhalation and for a time period which is a fraction of the duration of the inhalation. Thus, these patents also follow the reasoning that insufflation at the beginning of inhalation will effectively supply the respirating gas to the patient.

In yet another prior art system, a supplemental oxygen delivery system begins to deliver a steady flow of oxygen during a later stage of the exhalation interval and through an advanced stage of the inhalation interval of the breathing cycle and superimposes upon this steady flow of oxygen a peak pulse flow of oxygen at the beginning of inhalation. This is described in U.S. Pat. No. 4,686,974 to Sato et al. which discloses a breath-synchronized gas-insufflation device. This device includes a gas source, a valve, an insufflating device, a sensor, and an operational controller. The valve is connected to the gas source so as to regulate flow rate and duration of the gas flow from the gas source. The insufflating device is connected to the valve so as to insufflate the gas therefrom toward a respiratory system of a living body. The sensor is exposed to respiration of the living body and produces electric signals which must distinctively indicate an inhalation interval and an exhalation interval of the breathing cycle. The operational controller receives the electric signals from the sensor and produces control signals to the valve so that gas insufflation starts before the beginning of the inhalation interval and ends before termination of the inhalation interval while providing a short pulse-like peak flow of a large amount of the gas in an early stage of the inspiratory interval. Specifically, steady insufflation of the gas starts before the beginning of each inhalation and the pulse-like peak flow insufflation of the gas is superimposed on the steady insufflation for a short period of time after the beginning of the inhalation. An arbitrary time interval, based upon an average exhalation period and an average inhalation period, is chosen to trigger and end insufflation during the breathing cycle.

Although the prior art devices discussed hereinabove indeed conserve a treatment gas, such as oxygen, they fail to address the problem related to the changing respiratory needs of the patient that vary with different patient activity levels. When a patient requiring supplemental oxygen is at rest, relatively small quantities of oxygen are needed to maintain appropriate levels of oxygen concentration in the blood and thereby prevent what is termed “desaturation”. With an increase in the physical activity of a patient, larger quantities of oxygen are needed to maintain appropriate levels of oxygen concentration in the blood compared to when the patient is at rest.

Such systems are generally referred to as “on the go” systems and an example of such is U.S. Pat. No. 4,706,664 wherein Snook et al. discloses a pulse-flow supplemental oxygen apparatus which yields savings in oxygen while affording the patient the physiological equivalent of a prescribed continuous stream of oxygen. The apparatus includes a demand oxygen valve operated in a pulse mode by means of electronic control circuitry. Through an appropriate sensor, the electronic control circuitry monitors the patient's breathing efforts and gives a variable timed pulse of oxygen to increase the volume delivered to the patient during the very initial stage of each inhalation interval of the breathing cycle or breath. Pulse volume variability is based upon a measured parameter characterizing a plurality of the patient's preceding breathing cycles. The elapsed time interval of the patient's three preceding breathing cycles is measured to effectively measure the rate of the breathing cycles. These breath-characterizing parameters, together with data characterizing the prescribed continuous oxygen flow to be matched, enable the apparatus to give the desired dose variability.

In yet another example, U.S. Pat. No. 4,584,996 to Blum reveals a method and apparatus for intermittent administration of supplemental oxygen to patients with chronic lung dysfunction. The apparatus is programmable for administering the specific oxygen requirements of the patient and is responsive to changes in these oxygen requirements with increased patient activity. The patient's arterial blood oxygen level is measured while supplying oxygen to the patient during inspiration to determine the number of breathing cycles required to reach a first higher arterial blood oxygen level and is again measured without supplemental oxygen to determine the number of breathing cycles required to diminish the arterial blood oxygen level to a second, lower level. These two cycle numbers are utilized in an algorithm which is applied as a program to the apparatus having a breathing cycle sensor, a counter and control valve. The control valve provides a regulated flow of supplemental oxygen to a nasal cannula for a predetermined number of “ON” breathing cycles and to shut off the flow for a preset number of “OFF” breathing cycles sequentially and repetitively, thereby conserving oxygen while medically monitoring the patient's blood oxygen levels. The oxygen conservation features of this apparatus are further enhanced by turning off the oxygen flow during the exhalation interval of each breathing cycle throughout the “ON” breathing cycles. As the respiratory rate of the patient increases with patient activity, the duration of the “ON” and “OFF” periods changes accordingly.

In U.S. Pat. No. 4,686,975, Naimon et al. teaches a supplemental respiratory device that uses electronic components to intermittently regulate the flow of a respirable gas to a user on a demand basis. By monitoring small changes in the relative airway pressure, this respiratory device supplies gas only when an inhalation is detected. This respiratory device can also vary the duration of the gas delivery time to compensate for changes in the user's breath rate, thereby attempting to adjust for changes in the patient's respiratory needs based upon activity.

There are many other examples of “on demand” and “on the go” systems as many manufacturers are marketing oxygen conserver devices which are adapted to retrofit onto typical supplemental oxygen delivery systems that employ any type of oxygen source, such as portable oxygen tanks, oxygen concentrators or wall outlet supplies often utilized in hospitals. These oxygen conserver devices are adapted to be interposed between the oxygen source and a conventional nasal cannula apparatus. Medisonic U.S.A., Inc. of Clarence, N.Y., manufactures an oxygen conserver device entitled MedisO.sub.2 nic Conserver. It conserves oxygen by interrupting the flow of oxygen from the source to the patient during the exhalation interval of the patient's breathing cycle. Chad Therapeutics, Inc. of Chatsworth, Calif., manufacturers an oxygen conserver device bearing a registered trademark, Oxymatic® Electronic Oxygen Conserver. Chad's oxygen conserver eliminates oxygen waste during both the exhalation interval and the later portion of the inhalation interval of the breathing cycle. TriTec, Inc. of Columbia, Md., manufactures a demand oxygen cannula for portable oxygen systems that also responds to the negative pressure of inhalation. Smith-Perry Corporation of Surrey, British Columbia, Canada, manufactures The VIC (Voyager Intermittent Controller) Breathsaver that senses every breath of the patient and delivers a measured dose of oxygen only when the patient inhales. Pulsair, Inc. of Fort Pierce, Fla., manufactures an oxygen management system that delivers oxygen to the patient “on demand” at the initiation of inhalation. The Henry G. Dietz Co., Inc. of Long Island City, N.Y., manufactures an oxygen conserver device entitled Hala'tus 1 which conserves oxygen by sensing when inhalation takes places and delivers the oxygen only during inhalation.

It must be noted, however, that none of these oxygen conserver devices deliver oxygen to the patient during any stage of exhalation, and thus do not operate in accordance with the actual characteristics of the breathing cycle and the patient breathing system. In addition, none of these systems address the problems associated with maintaining an acceptable level of treatment gas in the patient under fault conditions, such as a loss of inhalation/exhalation data from the patient or a loss of power in the system.

### SUMMARY OF THE INVENTION

Wherefore, it is an object of the present invention to overcome the above noted drawbacks associated with the prior art treatment gas delivery systems.

Another object of the present invention is to provided a system and method which commences delivery of a treating gas, such as oxygen or room air with a concentrated or increased level of oxygen, to a patient during the final stage of the exhalation breath of the patient so that air, e.g., oxygen or room air with a concentrated or increased level of oxygen, is instantaneously available, at the nasal and/or mouth cavities of the patient, immediately prior to the time when the patient commences an inhalation breath. As a result of this, oxygen or room air with a concentrated or increased level of oxygen, is instantaneously available for inhalation at the beginning of the inhalation breath of the patient.

A further object of the present invention is to supply a bolus or pulse of a treating gas such as oxygen or room air with a concentrated or increased level of oxygen, directly to the nasal cavities and/or mouth cavity of the patient, immediately prior to the patient commencing an inhalation breath, so that this bolus or pulse of concentrated or increased level of oxygen is able to dilute the carbon dioxide concentration and increase the oxygen concentration of the air or gases contained within the nasal cavities and/or mouth cavity of the patient prior to the patient commencing his/her inhalation breath. This initial pulse or bolus of oxygen is effective in diluting the concentration of carbon dioxide contained within the nasal cavities, the mouth cavity, the larynx and/or the bronchi of the patient and also enriches or increases the oxygen concentration of the gases contained in those areas immediately prior to the patient commencing his/her inhalation breath. Such dilution of the concentration of carbon dioxide and enrichment of the concentration of oxygen will, in turn, assist in maintaining the blood oxygen saturation level of a patient at approximately 92% to 93%.

Yet another object of the present invention is to provide a failure mechanism which is designed to provide an adequate supply of oxygen, e.g., room air with a concentrated or increased level of oxygen, to the patient in the event that the system or method is unable to detect, determine or sense breathing of the patient. The failure mechanism ensures that the treatment gas saturation level or the blood oxygen saturation level of the patient remains adequately saturated with oxygen, e.g., at a blood saturation oxygen level of approximately 92% to 93% during use of the system.

A still further object of the present invention is to provide a system and method which delivers treating gas such as oxygen at a rate of 20 cycles per minute, or some other desired delivery rate, in the event the system and method fail to detect, determine or sense breathing of a patient. For example, the system or method will deliver oxygen to the patient for an interval of about 1 second or so and then interrupt the flow of oxygen to the patient for an interval of about 2 seconds or so, and then again deliver oxygen to the patient for a further interval of about 1 second or so and then interrupt the flow of oxygen to the patient flow for an interval of about 2 seconds or so, and so forth, thereby providing 20 oxygen delivery cycles per minute to the patient.

Yet another object of the present invention is to provide the system and method with a default mode of operation so that, in the event that a malfunction of the power source or some other component of the conserver, the oxygen control valve is controlled to its normally open default state to supply a continuous flow of oxygen, of some other treating gas to the patient, and ensure that the patient receives a continuous and constant supply of oxygen or some other treating gas and is maintained adequately saturated even during malfunction of one or more components of the system or method.

The present invention also relates to a system for conserving supply of a treatment gas to a patient, the system comprising: a source of the treatment gas; a sensing device for sensing a breathing cycle of a patient; a conserver for controlling intermittent supply of the treatment gas to the patient in response to the sensed breathing cycle; wherein the conserver operates in a first mode when the sensing device senses the breathing cycle to supply the treatment gas to the patient in a first intermittent cycle coordinated with the breathing cycle; and the conservator operates in a second mode, when the sensing device is unable to sense the breathing cycle, in which the conserver supplies the treatment gas to the patient at a second intermittent cycle determined independently of the patient breathing cycle where the second intermittent cycle is selected to overlap an assumed patient breathing cycle such that at least a desired amount of the treatment gas is supplied to the patient.

The present invention also relates to a method for maintaining a predetermined level of a treatment gas in a patient while conserving use of the treatment gas by delivering the treatment gas from a treatment gas source and to a patient interface via a conserver connected between the treatment gas source and the patient interface, the method comprising the steps of: sensing parameters of a breathing cycle of the patient; controlling operation of the conserver according to the sensed parameters of the breathing cycle so that the conserver operates in one of a first mode and a second mode; during the first mode, when at least one parameter of the breathing cycle is sensed, the conserver supplying the treatment gas to the patient in a first intermittent cycle coordinated with the patient breathing cycle, and during the second mode, when the conserver is unable to sense at least one parameter of the breathing cycle, the conserver supplying the treatment gas to the patient in a second intermittent cycle determined independently of the patient breathing cycle, in which the second intermittent cycle is selected to overlap an assumed patient breathing cycle such that at least a desired level of the treatment gas is maintained in the patient.

### BRIEF DESCRIPTION OF THE DRAWINGS

The invention will now be described, by way of example, with reference to the accompanying drawings in which:

FIG. 1 is a diagrammatic representation of a conventional treatment gas apparatus of the prior art;

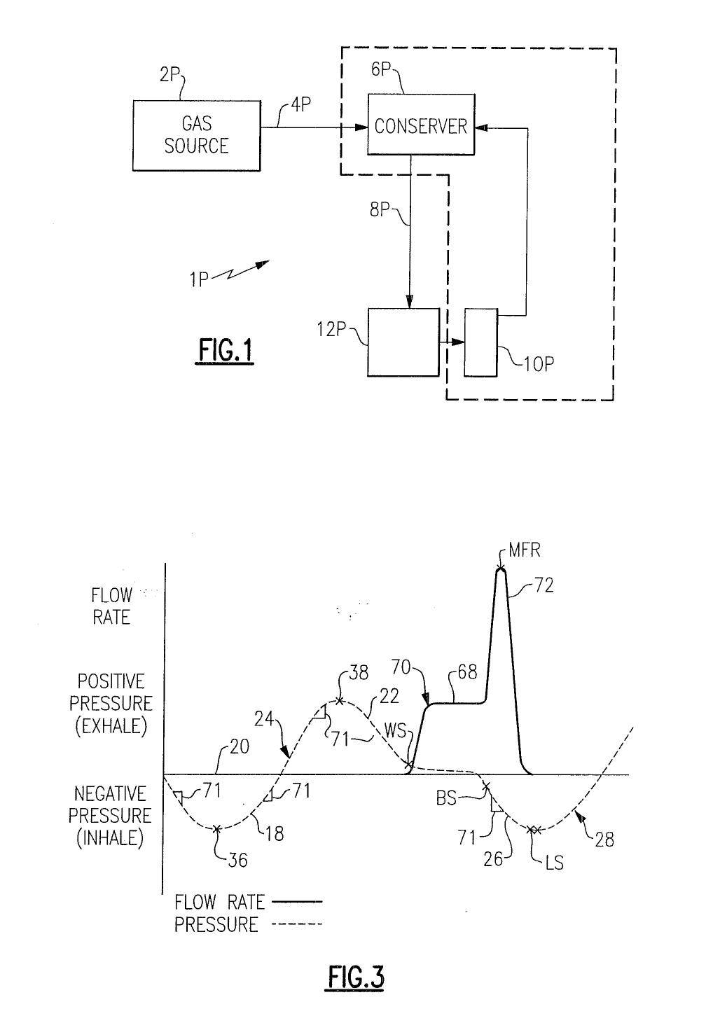

FIG. 2 is a schematic diagram of a first exemplary embodiment of an intermittent gas-insufflation apparatus of the present invention shown operably connected to and between a breathing patient and a source of treatment gas;

FIG. 3 is a graph illustrating a flow rate profile of the treatment gas being delivered to the patient superimposed over an inhalation interval and an exhalation interval of an immediate breathing cycle and a subsequent inhalation interval of a successive breathing cycle;

FIG. 4 is a schematic diagram of a power source accompanied by an electrical schematic diagram which is incorporated into the intermittent gas-insufflation apparatus of the present invention;

FIGS. 5A and 5B are schematic diagrams of a sensor, a reference voltage generator, a controller in a form of a microprocessor and a valve assembly including a first solenoid valve and a second solenoid valve which are incorporated into the intermittent gas-insufflation apparatus of the present invention;

FIGS. 6A and 6B are flow diagrams of the software program which shows the controller of the intermittent gas-insufflation apparatus of the present invention;

FIG. 7 is a flow chart for a “failsafe” mechanism to ensure an adequate level of treatment gas to the patent; and

FIG. 8 is a diagrammatic illustration of the operation of the present invention for providing an acceptable level of treatment gas to the patient under fault conditions, such as a loss of inhalation/exhalation data from the patient or a loss of power in the system.

### DETAILED DESCRIPTION OF THE INVENTION

The present invention is directed at maximizing conservation of the delivery of a treatment gas to a patient while, at the same time, maintaining the patient adequately saturated during, for example, a loss of respiratory data from the patient or a loss of power in the system. Systems for doing so are frequently referred to as “intermittent gas-insufflation” systems wherein the term “gas-insufflation” refers generally to the delivery of a treatment gas, such as oxygen or a carrier gas carrying suspended particles or droplets of medication of some other treatment fluid(s) or material(s), to a patient to be breathed in by the patient. The term “intermittent”, in turn, refers to such systems that do not deliver a continuous flow of treatment gas, but instead deliver the treatment gas only during those phases of the breathing cycle or during those demand periods when the delivery would be advantageous, thereby reducing the consumption of the treatment gas while providing adequate levels of the treatment gas to a patient.

The treatment gas may be, for example, oxygen, room air having an elevated or increased concentration of oxygen, (e.g., a diluted concentration of nitrogen) nitrous oxide, or any form of gas having droplets or particles of a medication or some other drug in suspension, and so on, all of which are herein after referred to herein generically as a “treatment gas”. It should be noted, however, that the following discussions will often refer to the treatment gas as “oxygen” or concentrated “room air”, and it must be understood that oxygen is selected for the following exemplary embodiments because oxygen is one of the more common treatment gases administered to a patient.

Therefore, for purposes of the following discussions and in the case of oxygen or room air having an elevated or increased concentration of oxygen as the treatment gas, the treatment gas is supplied to maintain the blood oxygen saturation level of a patient at approximately 92% to 93%. The inventor has determined that by commencing delivery of oxygen or room air with an increased concentration of oxygen as the treatment gas to the patient, slightly or immediately prior to the patient completing his/her exhalation breath, i.e., during the last 10% or so of the patient's exhalation breath, this ensures that an adequate supply of oxygen is delivered to and available for inhalation by the patient immediately prior to the moment when the patient commences inhalation. That is, immediately prior to the moment when the patient commences inhalation, the oxygen or room air with an increased concentration of oxygen is supplied to and fills the nasal cavities and/or the mouth cavity of the patient for inhalation by the patient.

In addition, the present invention is directed at supplying the oxygen to those cavities or regions of the patient, immediately prior to the patient commencing inhalation, so that the oxygen, which is delivered by the conserver to the patient at a desired pressure and flow rate, adequately dilutes and diffuses the concentration of carbon dioxide contained in the exhaled breath of the patient while, at the same time, enriches the concentration of oxygen available for inhalation by the patient during his/her next breath.

In this regard, room air ordinarily has an oxygen content of about 22% while the gas being exhausted or exhaled from the lungs of a patient typically has an oxygen content of about 17 to 18%, for example. In particular, the exhaled gases from the lungs which have not been completely exhaled from and are still located within the trachea, the bronchi, the nasal cavities and/or the mouth cavity of the patient typically have an oxygen content of 17 to 18%. As these gases have not been completely exhaled, these same gases are the first gases that are initially inhaled by the patient during the next subsequent breath.

The conserver and method of the present invention are specifically directed at supplying an initial pulse or bolus (e.g., approximately 35 cc) of oxygen or room air with an increased concentration of oxygen which is specifically designed to diffuse the concentration of carbon dioxide and other gases contained within breathing cavities and/or areas and enrich the concentration of oxygen to about 20% to 21% or so, for example, depending upon the flow rate of the oxygen, the supply pressure of the oxygen, etc.

The inventor has, therefore, appreciated that during the transition from exhalation to inhalation, the patient will generally initially inhale the last portion of breath (e.g., approximately 150 cc or so depending upon the physical characteristics of the patient) that was in the process of being exhaled by the patient but was not expelled from the lungs, the trachea, the nasal and/or mouth cavities. Accordingly, the present invention is directed at diluting the concentration of the carbon dioxide and increasing the concentration of oxygen contained in the exhaled but not expelled breath of the patient so that, upon commencing inhalation by the patient, the concentration of carbon dioxide and oxygen initially inhaled by the patient during the first 10% of the inhalation breath are optimized. At assist with this, the conserver controls a flow valve which, when open, releases the treatment gas which accumulates in a bolus chamber (e.g., a 35 cc bolus chamber) and supplies this treatment gas directly to the nasal and/or mouth cavities. This bolus or initial pulse of oxygen is designed to dilute and diffuse the concentration of carbon dioxide while, at the same time, enrich the concentration of oxygen prior to the patient inhaling the next subsequent breath.

First considering an exemplary intermittent gas-insufflation apparatus in which the present invention can be and is implemented, such a system is adapted to be disposed between and in fluid communication with a source of gaseous treatment fluid, such oxygen or air with an enriched oxygen content, and a breathing patient. The intermittent gas-insufflation apparatus is operative to insufflate a quantity of the treatment gas, e.g., oxygen, into an entrance of a respiratory system of the patient after an inhalation interval and during an exhalation interval of an immediate breathing cycle and into a subsequent inhalation interval of a successive breathing cycle of the patient. For purposes of explaining the intermittent gas-insufflation apparatus of the present invention, it would be beneficial to discuss several terms used throughout the description of the exemplary embodiments of the present invention to better understand the operation and components thereof. Quotation marks are employed to highlight the first usage of each term in the explanation discussed below.

A “breathing cycle” occurs when the patient first inhales and then exhales; the breathing cycle commences when the patient begins to inhale and terminates when the patient completes exhalation. As a result, a breathing cycle consists of an “inhalation interval” and an “exhalation interval” which follows the inhalation interval. A convention used for explanation purposes only of the exemplary embodiments of the present invention is that the inhalation interval is sensed by detection of “negative pressure values” relative to an ambient pressure environment which is generated as the patient inhales and the exhalation interval is sensed by detection of “positive pressure values” relative to the ambient pressure environment which is generated as the patient exhales. Particularly useful for explanation of the first exemplary embodiment of the intermittent gas-insufflation apparatus of the present invention is a “negative peak pressure value” which occurs at the lowest pressure value detected during the inhalation interval of the immediate breathing cycle and a “positive peak pressure value” which occurs at the highest pressure value detected during the exhalation interval of the immediate breathing cycle. These negative and positive peak pressure values are employed for the operation of the first exemplary embodiment of the intermittent gas-insufflation apparatus of the present invention.

Furthermore, “immediate breathing cycle” and “successive breathing cycle” are used herein as a convention only to explain the operation of the present invention. As suggested by the terms themselves, the immediate breathing cycle is the breathing cycle in which the patient is currently breathing and the successive breathing cycle follows the immediate breathing cycle. In reality, once the “immediate breathing cycle” terminates, the “successive breathing cycle” now becomes the immediate cycle and the immediately terminated cycle then becomes the preceding breath cycle. It would be understood by one of ordinary skilled in the art that a patient breaths only during the immediate breathing cycle. Additionally, “changes in breathing pressure” can be construed as either actual changes of breathing pressure or changes in the rate of breathing pressure.

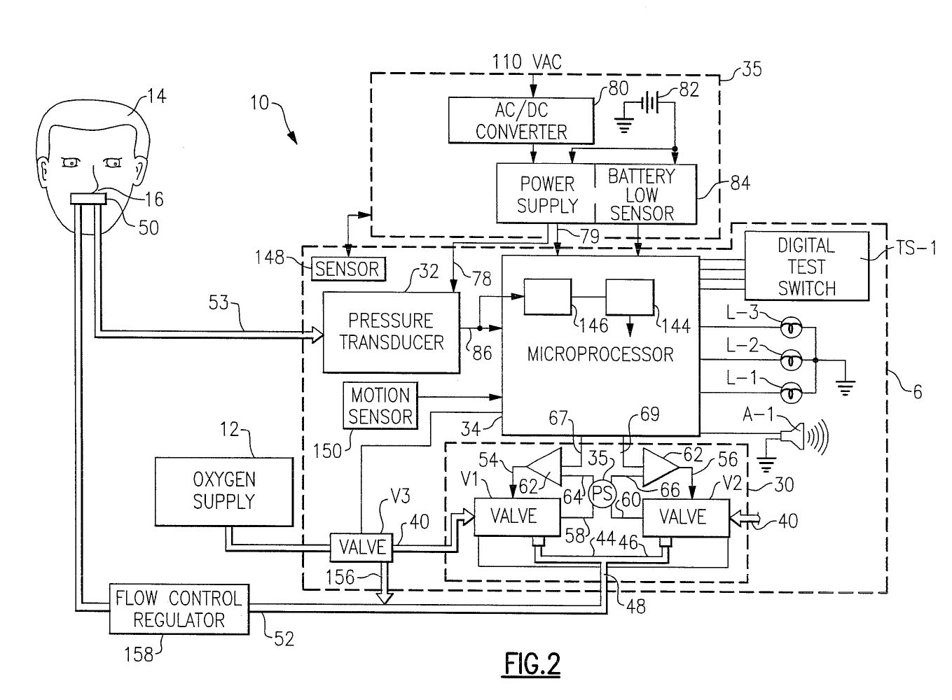

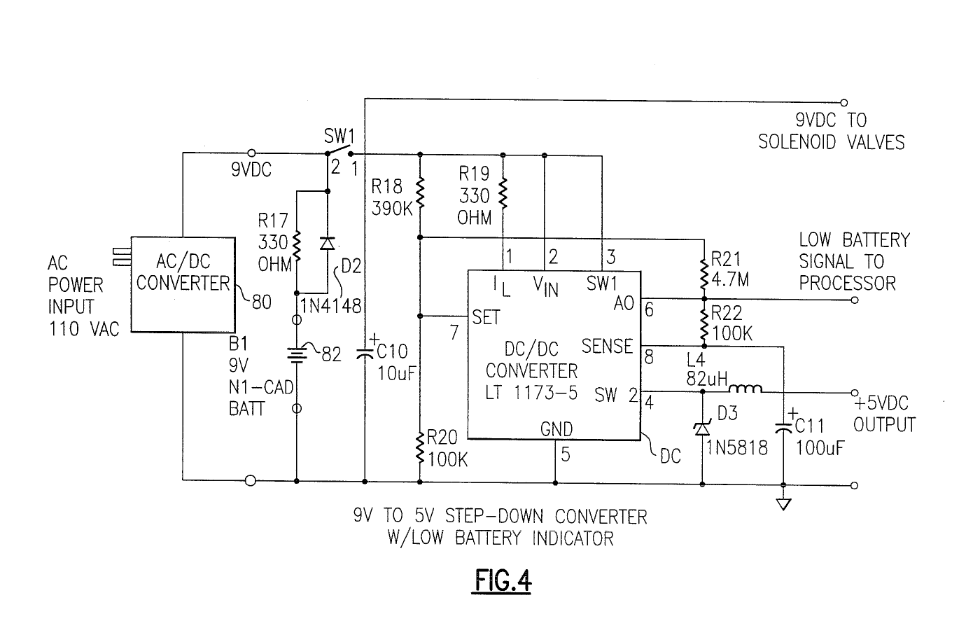

A first exemplary embodiment of an intermittent gas-insufflation apparatus 10 of the present invention with a conserver 6 of the present invention is generally described with reference to FIGS. 2 and 3. Intermittent gas-insufflation apparatus 10 is adapted to be disposed between and in fluid communication with an oxygen supply or some other source 12 of treatment gas and a breathing patient 14. In a present embodiment of the invention, it is preferred that the treatment gas is oxygen although the treatment gas could also be selected from a group consisting of air, nitrous oxide, ether and other gases normally administered to human beings and animals. Intermittent gas-insufflation apparatus 10 is operative to insufflate a quantity of the treatment gas into an entrance 16 of a respiratory system of patient 14. Typically, entrance 16 is a nose or mouth of patient 14, although, in some instances, entrance 16 could be both the nose and the mouth of patient 14. With reference to FIG. 3, the quantity of the treatment gas (solid line) is continuously insufflated after an inhalation interval 18 (dotted line below base line 20) and during an exhalation interval 22 (dotted line above base line 20) of an immediate breathing cycle 24. As stated above, immediate breathing cycle 24 is inhalation interval 18 plus exhalation interval 22. Insufflation of the treatment gas continues into a subsequent inhalation interval 26 of a successive breathing cycle 28 of patient 14.

It must be noted that the source 12 of treatment gas may be implemented in a number of different ways without departing from the invention as described herein. For example, and assuming for purposes of illustration that the treatment gas is oxygen or air with enriched levels of oxygen, the source 12 may be a liquid or gaseous oxygen source, such as a tank of liquid oxygen or pressurized gaseous oxygen or a tank of mixed gases in liquid or gaseous form, or may be a concentrator, as is often employed when the treatment gas is oxygen enriched air. As is well understood, a conventional concentrator employs molecular sieves to selectively absorb certain gases from the ambient air, such as nitrogen, so that the resulting treatment gas has an increased proportional oxygen level compared to the other remaining gases in the mixture.

Again, with reference to FIG. 2, intermittent gas-insufflation apparatus 10 includes a valve assembly 30, a pressure transducer sensor 32, a microprocessor controller 34 and a power source 35 which is operative to energize valve assembly 30, sensor 32 and controller 34. Valve assembly 30 is disposed between and in fluid communication with source 12 of treatment gas and entrance 16 into the respiratory system of patient 14. Valve assembly 30 is operative to actuate between a closed state and an opened state. In the closed state, fluid communication is interrupted so that the treatment gas is prevented from flowing from source (e.g., oxygen supply) 12 of treatment gas to entrance 16 into the respiratory system of patient 14. In the opened state, fluid communication is established so that the treatment gas flows from source 12 of treatment gas to entrance 16 into the respiratory system of patient 14.

For purposes of the present invention and the descriptions thereof, valve assembly 30, sensor 32 and controller 34 may be regarded as comprising a conserver 6 of the present invention. As will be described in the following, the conserver 6 of the present invention includes fundamental features and aspects of operation that distinguish the conserver 6 over the conserver 6P of the prior art and enable the conserver 6, according to the present invention, to perform the desired functions of the present invention.

Sensor 32, in a form of a pressure transducer, is in fluid communication with entrance 16 of the respiratory system of patient 14 and is operative to detect changes in breathing pressure (represented by the dashed sinusoid line in FIG. 3) of breathing patient 14 relative to an ambient pressure environment as patient 14 breaths. Although not by way of limitation, it is preferred, for the first exemplary embodiment of the present invention, that the detected changes in breathing pressure are actual changes in the breathing pressure. Specifically, sensor 32 is operative to detect changes in breathing pressure throughout inhalation and exhalation intervals 18 and 22, respectively, of immediate breathing cycle 24 of patient 14. Sensor 32 is further operative to generate sensor signals characteristic of the changes in breathing pressure of immediate breathing cycle 24. These changes in breathing pressure, plotted as a function of time (base line 20), is represented by the dashed sinusoidal line shown in FIG. 3.

Controller 34 in a form of a microprocessor is coupled to and between sensor 32 and valve assembly 30 (FIG. 2) and is operative to receive and process the sensor signals to determine a negative peak pressure value 36 (FIG. 3) which occurs during inhalation interval 18 of the immediate breathing cycle 24 and a positive peak pressure value 38 which occurs during exhalation interval 22 of the immediate breathing cycle 24. Controller 34 is responsive within exhalation interval 22 of immediate breathing cycle 24 when a first predetermined percentage of positive peak pressure value 38 is achieved which is discussed in more detail below. Upon achieving the first predetermined percentage of positive peak pressure value 38, valve assembly 30 is actuated to the opened state so that the treatment gas flows from source 12 of treatment gas to entrance 16 into the respiratory system of patient 14 during exhalation interval 22 of immediate breathing cycle 24 and during inhalation interval 18 of successive breathing cycle 28. Controller 34 is further responsive within subsequent inhalation interval 26 of successive breathing cycle 28, when a second predetermined percentage of negative peak pressure value 36 is achieved to actuate valve assembly 30 to the closed state so that the treatment gas is prevented from flowing from source 12 of treatment gas to entrance 16 into the respiratory system of patient 14.

Controller 34 is further responsive within subsequent inhalation interval 26 of successive breathing cycle 28 when a third predetermined percentage of negative peak pressure value BS is achieved to further actuate valve assembly 30 into an enhanced opened state. In the enhanced opened state, an additional quantity of treatment gas flows from source 12 of treatment gas to entrance 16 into the respiratory system of patient 14 after exhalation interval 22 of immediate breathing cycle 24 and before a remaining portion of subsequent inhalation interval 26 of successive breathing cycle 28. Alternatively, a single valve assembly 30 could be actuated into the enhanced opened state during exhalation interval 22 of immediate breathing cycle 24, if desired.

The first, second and third predetermined percentages are determined clinically by a clinician for each individual patient. Preferably, at least the first and second predetermined percentages are tailored to respiratory needs of each individual patient although the third predetermined percentage can be tailored to respiratory needs of each individual patient. Thus, the intermittent gas-insufflation apparatus of the present invention is tailored to the patient's particular supplementary oxygen needs. Factors which might be considered by the clinician are weight, height, physical condition, severity of lung dysfunction and the like. The first and second predetermined percentages are selected from a range of 10% to 80%. The first and second predetermined percentages are typically different from one another although they could be the same. The first and second predetermined percentages are selected from a range of 10% and 80% inclusive. Preferably, the first predetermined percentage is 25%; the second predetermined percentage is 33.3%. The third predetermined percentage is selected from a range of 1% and 25% inclusive as long as it is less than the second predetermined percentage. Preferably, the third predetermined percentage is 12.5%.

For the first exemplary embodiment of intermittent gas-insufflation apparatus 10 of the present invention, valve assembly 30 includes a first solenoid valve V1 and a second solenoid valve V2. First solenoid valve V1 is operative between a first closed state and a first opened state; second solenoid valve V2 is operative between a second closed state and a second opened state. Each of first and second solenoid valves V1 and V2 is independently connected in fluid communication to and between source 12 of pressurized gas and entrance 16 to the respiratory system of patient 14. Gas supply tubing 40 connects first and second solenoid valves V1 and V2 to source 12 of pressurized treatment gas. Respective ones of valve tubings 44 and 46 connect first and second solenoid valves V1 and V2 to a manifold 48. Manifold 48, in turn, is connected to a nasal cannula 50 via a single gas delivery tube 52. First and second solenoid valves V1 and V2 are independently connected electrically to controller 34 via line 54 and 56 and to power source 35 via lines 58 and 60. First and second solenoid valves V1 and V2 have a valve driver 62 interposed in respective lines 54 and 56 and each valve driver 62 is electrically connected to power source 35 via respective lines 64 and 66. Each valve driver 62 is electrically connected to controller 34 via lines 67 and 69.

Nasal cannula 50, gas delivery tube 52 and a sensing tube 53 are components of a conventional cannula structure commonly known in the art. In brief, nasal cannula 50 is sized and adopted to be received by and secured proximate to the entrance of the respiratory system of the breathing patient 14. Nasal cannula 50 has a septum, a partition or some other dividing or separating structure (not shown) dividing or separates nasal cannula 50 into a gas delivery conduit and a sensing conduit which are isolated from fluid communication with one another. The gas delivery conduit is in fluid communication with valve assembly 30 via gas delivery tube 52 and the sensing conduit is in fluid communication with sensor 32 via sensing tube 53. Thus, nasal cannula 50, sometimes referred to as a divided or split cannula, can both detect changes in breathing pressure and deliver oxygen to the patient simultaneously.

Again, with reference to FIGS. 2 and 3, first solenoid valve V1 is operative to actuate from the first closed state to the first opened state during exhalation interval 22 of immediate breathing cycle 24 and from the first opened state to the first closed state at a later stage “LS” of subsequent inhalation interval 26 of successive breathing cycle 28. Thus, the treatment gas flows (solid line) as shown during exhalation interval 22 of an immediate breathing cycle 24 which begins at a waning stage “WS” of exhalation interval of the immediate breathing cycle. Waning stage “WS” represents the first predetermined percentage multiplied by a positive peak pressure value 38. When in the first opened state, the treatment gas flow builds to a steady state flow as shown by a flat solid line portion 68 of flow trace 70. Meanwhile, second solenoid valve V2 is actuated from the second closed state to the second opened state at approximately a beginning stage “BS” of a subsequent inhalation interval 26 of successive breathing cycle 28 thereby causing the enhanced opened state of valve assembly 30. Beginning stage “BS” represents the third predetermined percentage multiplied by the peak negative pressure value of the immediate breathing cycle which is used in the subsequent inhalation interval. In the second opened state of second solenoid valve V2, the additional treatment gas flows as a high flow-rate pulse reflected by the spiked solid line portion 72 of flow trace 70. The second solenoid valve V2 is actuated from the second opened state to the second closed state at later stage “LS” of subsequent inhalation interval 26 of successive breathing cycle 28. Later stage “LS” represents the second predetermined percentage multiplied by the negative peak pressure value of the immediate breathing cycle. Thus, although not by way of limitation, first solenoid valve V1 and second solenoid valve V2 actuate to their respective closed states simultaneously. Preferably, later stage “LS” occurs before the negative peak pressure value of the subsequent inhalation interval. Furthermore, first solenoid valve V1 and second solenoid valve V2, respectively, actuate to the first closed state and the second closed state when the second predetermined percentage of negative peak pressure value 36 is achieved. In any event, treatment gas flows at a flow rate selected from a flow rate range of between 0.5 liters per minute and 12 liters per minute inclusive.

One of ordinary skill in the art would appreciate that the intermittent gas-insufflation apparatus of the present invention operates within its own operating cycle which is hereinafter deemed an “insufflation operating cycle”. The insufflation operating cycle begins at the negative peak pressure value of the inhalation interval of the immediate breathing cycle, continues through the exhalation interval of the immediate breathing cycle and terminates before the negative peak pressure value of a subsequent inhalation interval of the successive breathing cycle. A skilled artisan would understand that the insufflation operating cycle of the present invention is considered to be phase shifted forward by 90 degrees relative to the patient's normal breathing cycle. Additionally, one of ordinary skill in the art would appreciate that the present invention generates these negative and positive peak pressure values to activate the present invention during the immediate breathing cycle and utilizes reference pressures from the immediate breathing cycle to de-activate the present invention during the successive breathing cycle. Moreover, it is appreciated that the intermittent gas insufflation apparatus of the present invention detects changes in pressure, utilizes these detected pressure changes for delivery of the treatment gas, and then commences delivery of the treatment gas to the patient within the patient's immediate breathing cycle, which has not heretofore been accomplished by any of the prior art gas insufflation devices.

It follows from the first exemplary embodiment of intermittent gas-insufflation apparatus 10 of the present invention, a method can be employed for intermittently insufflating a treatment gas from a pressurized treatment gas source 12 into entrance 16 of a respiratory system of a breathing patient 14 after inhalation interval 18 and during exhalation interval 22 of immediate breathing cycle 24 and into subsequent inhalation interval 26 of successive breathing cycle 28. The first step of this method is determining the negative peak pressure value which occurs during inhalation interval 18 of immediate breathing cycle 24. The next step is determining the positive peak pressure value which occurs during exhalation interval 22 of immediate breathing cycle 24. The next step includes commencing delivery of the treatment gas to entrance 16 of the respiratory system of patient 14 during exhalation interval 22 of immediate breathing cycle 24 when the first predetermined percentage of positive peak pressure value 38 is achieved. The next step includes continuing delivery of the treatment gas to entrance 16 of the respiratory system during subsequent inhalation interval 26 of successive breathing cycle 28. The final step is ending delivery of the treatment gas to the respiratory system during subsequent inhalation interval 26 of successive breathing cycle 28 when a second predetermined percentage of negative peak pressure value 36 is achieved. Furthermore, the step of commencing delivery of additional treatment gas to entrance 16 of the respiratory system of patient 14 during subsequent inhalation interval 26 of successive breathing cycle 28 when a third predetermined percentage of negative peak pressure value 36 is achieved can also be added after continuing delivery of the treatment gas to entrance 16 of the respiratory system during subsequent inhalation interval 26 of successive breathing cycle 28.

A second exemplary embodiment of an intermittent gas-insufflation apparatus employs a variable orifice valve, such as a conventional tapered-needle valve. This second exemplary embodiment of the intermittent gas-insufflation apparatus employs the same general operational principles of the first exemplary embodiment of the intermittent gas-insufflation apparatus 10 except that a different type of valve is used in lieu of the first and second solenoid valves. Also, the second exemplary embodiment of the intermittent gas-insufflation apparatus requires some modification to the software program which controls controller 34. With modification to the software program, controller 34 is now operative to receive and process the sensor signals generated by sensor 32 during immediate breathing cycle 24 to calculate how much of a quantity of the treatment gas is required by the breathing effort of patient 14. For the second exemplary embodiment of the present invention, it is preferred that sensor 32 detects a rate of change of the breathing pressure of the patient. Controller 34 is responsive to the sensor signals to actuate valve assembly 30 into the opened state so that the calculated quantity of treatment gas flows from source 12 of treatment gas to entrance 16 into the respiratory system of patient 14 during exhalation interval 22 of immediate breathing cycle 24 and into subsequent inhalation interval 26 of successive breathing cycle 28. Controller 34 is further responsive to actuate valve assembly 30 into the closed state during subsequent inhalation interval 26 of successive breathing cycle 28 when the calculated quantity of treatment gas is delivered to entrance 16 into the respiratory system of patient 14. It is preferred that valve assembly 30 actuates to the closed state before the negative peak pressure value of the subsequent inhalation interval of the successive breathing cycle is achieved.

The calculated quantity of the treatment gas to be delivered to the patient is predicated upon the immediate breathing cycle. So, as the patient's respiratory needs change, for example, as a result of increased physical activity, the calculated quantity of the treatment gas will also increase. Correspondingly, when the patient's physical activity decreases, changes in breathing pressure will be detected and the calculated quantity of treatment gas will also decrease.

The rate of change of pressure can be calculated by dividing a difference between two detected pressure values by a difference of respective times during which the pressure valves were detected. This is illustrated in FIG. 3 by angles 71. A skilled artisan would appreciate that this is a calculation of the “slope” of flow trace 70. Note that the rate of change of pressure can be calculated during the inhalation interval of the immediate breathing cycle, during the exhalation interval of the immediate breathing cycle or even during the subsequent inhalation interval of the successive breathing cycle.

Additionally, controller 34 is further operative to determine a flow rate profile of the calculated quantity of the treatment gas for continuous flow thereof to entrance 16 into the respiratory system of the breathing patient during exhalation interval 22 of immediate breathing cycle 24 and subsequent inhalation interval 26 of successive breathing cycle 28. By way of example only and not of limitation, the flow rate profile is illustrated by the solid line flow trace 70 shown in FIG. 3. Since modification of the software program can determine the configuration of the flow rate profile as desired, the flow rate profile is selected from a group consisting of a constant flow rate profile as illustrated by flat solid line portion 68 of flow trace 70, a variable flow rate profile illustrated as the spiked solid line portion of flow trace 70 or a combination the fixed and the variable flow rate profile as illustrated in FIG. 3. Since the rate of change of pressure can be detected within the subsequent inhalation of the successive breathing cycle while the treatment gas is flowing to the entrance of the respiratory system of the patient, the flow rate profile of the flowing treatment gas can be instantly changed to facilitate complete and timely delivery of the calculated quantity of the treatment gas to the patient, if desired. This feature of the present invention has not heretofore been incorporated into any prior art. Obviously, the flow rate profile could be instantly modified, if desired, at any time during which the treatment gas is being delivered, i.e., during the exhalation interval of the immediate breathing cycle and the subsequently inhalation interval of the successive breathing cycle.

By way of example only, a maximum flow rate “MFR” of the calculated quantity of treatment gas flowing into entrance 16 of the respiratory system of the breathing patient during exhalation interval 22 of immediate breathing cycle 24 occurs shortly after beginning stage “BS” of inhalation interval 18 of the subsequent breathing cycle. Preferably, the flow rate profile of the treatment gas includes a flow rate range having a minimum flow rate of 0.5 liters per minute and a maximum flow rate of 12.0 liters per minute.

It follows from the second exemplary embodiment of the intermittent gas insufflation apparatus of the present invention, a method is employed for intermittently insufflating the treatment gas from the pressurized treatment gas source and into an entrance of a respiratory system of the breathing patient after the inhalation interval and during the exhalation interval of the immediate breathing cycle and into the subsequent inhalation interval of the successive breathing cycle. The first step is calculating the quantity of the treatment gas required to be delivered to entrance 16 of the respiratory system of patient 14 during one of the inhalation interval 18 and the exhalation interval 22 of immediate breathing cycle 24. The next step is commencing delivery of the calculated quantity of the treatment gas to entrance 16 of the respiratory system of patient 14 during exhalation interval 22 of immediate breathing cycle 24. The next step includes continuing delivery of the calculated quantity of the treatment gas to entrance 16 of the respiratory system of patient 14 into subsequent inhalation interval 26 of successive breathing cycle 28. The next step is ending delivery of the calculated quantity of the treatment gas to the respiratory system of patient 14 when delivery is complete during subsequent inhalation interval 26 of successive breathing cycle 28. It is preferred that a step of determining a desired flow rate profile for the delivery of the quantity of the treatment gas occurs simultaneously with the step of calculating the quantity of the treatment gas required to be delivered to entrance 16 of the respiratory system of patient 14 during one of inhalation interval 18 and exhalation interval 22 of immediate breathing cycle 24. It is also preferred that the step of delivering a maximum flow rate of the desired flow rate profile shortly after the beginning stage “BS” of subsequent inhalation interval 26 of successive breathing cycle 28. Of course, it is preferable to include a step of repeating the steps of this method for each series of consecutive immediate and successive breathing cycles.

A third exemplary embodiment of an intermittent gas-insufflation apparatus of the present invention incorporates valve assembly 30 which includes a shape-memory alloy-film actuated valve (commonly referred to as a microflow valve). This third exemplary embodiment of the intermittent gas insufflation apparatus of the present invention employs the same operational principles of the embodiments described above except that minor modifications of the software program controlling controller 34 must be made. As with any conventional shape-memory alloy-film actuated valve, actuating this valve can be controlled whereby the opened state can be varied, as dictated by the software program, as the treatment gas flows from the source to the patient. Thus, flow rate of the treatment gas can be precisely controlled at any time during delivery of the treatment gas to the patient.

Given the three exemplary embodiments of the intermittent gas insufflation apparatus of the present invention described above, one of ordinary skill in the art would appreciate the advancement made in the art. Particularly, the intermittent gas insufflation apparatus of the present invention includes the controller coupled to and between the sensor and the valve assembly which is operative to receive and process the sensor signals generated during either the inhalation interval of the immediate breathing cycle, the exhalation interval of the immediate breathing cycle or the inhalation and exhalation intervals of the immediate breathing cycle. Although not by way of limitation, valve assembly actuates to the opened state at waning stage “WS” of the exhalation interval of the immediate breathing cycle and actuates to the closed state during later stage “LS” of the subsequent inhalation interval of the successive breathing cycle. Furthermore, the intermittent gas insufflation apparatus of the present invention employs a method for intermittently insufflating a treatment gas from a pressurized treatment gas source and into an entrance of a respiratory system of a breathing patient. The first step includes generating sensor signals during either of the inhalation interval of the immediate breathing cycle, the exhalation interval of the immediate breathing cycle or both the inhalation and exhalation intervals of the immediate breathing cycle. The next step includes processing the sensor signals during either the inhalation interval of the immediate breathing cycle, the exhalation interval of the immediate breathing cycle or both of the inhalation and exhalation intervals of the immediate breathing cycle to determine the quantity of the treatment gas to be delivered to the entrance of the respiratory system of the patient. The next step is then commencing delivery of the quantity of treatment gas to the entrance of the respiratory system of the patient during the exhalation interval of the immediate breathing cycle. The following step is continuing delivery of the quantity of the treatment gas to the entrance of the respiratory system of the patient into the subsequent inhalation interval of the successive breathing cycle. The next step is ending delivery of the quantity of the treatment gas to the respiratory system of the patient during the subsequent inhalation interval of the successive breathing cycle.

A skilled artisan would comprehend that the valve assembly can employ any type of valve, conventional or otherwise. Depending upon the needs of the patient, the valve assembly could employ a single solenoid valve, a single stepped solenoid valve, a single proportional valve or a single shape-memory alloy-film actuated valve. Also, for any of the exemplary embodiments described herein, the present invention could incorporate an arrangement of solenoid valves, an arrangement of stepped solenoid valves, an arrangement of proportional valves, an arrangement of shape-memory alloy-film actuated valves and even an arrangement of any combination of these types of valves. Furthermore, the present invention could operate with the valve or valves normally in the opened state or normally in the closed state. Valves in the normally opened state would provide a “fail-safe” feature for the valve assembly whereby, for example, in the event of a power source failure, the valve or valves of the valve assembly would automatically actuate to the opened state. Thus, even without a power source, the patient would continue to receive oxygen at a default rate of flow, preferably about 2 liters per minute or some other desired flow rate.

Additionally, the intermittent gas insufflation apparatus of the present invention could be used with a blood-oxygen concentration device to maintain an appropriate blood-oxygen concentration in a patient's blood stream. For example, with an oximeter operably connected to a patient's ear, the software program could again be modified so that the quantity of oxygen to be delivered to the patient is based upon feedback from the oximeter concerning the patient's blood-oxygen concentration or content. Thus, a method is employed for maintaining at least a threshold amount of blood-oxygen concentration in the patient receiving supplemental oxygen from a supplemental oxygen delivery system. The first step includes monitoring the amount of blood-oxygen concentration in the patient. The next step is determining if the amount of blood-oxygen concentration in the patient is below the threshold amount of blood-oxygen concentration. The next step is activating the supplemental oxygen delivery system until the amount of blood-oxygen concentration is at least the threshold amount of blood-oxygen concentration for the patient.

Operation of the System

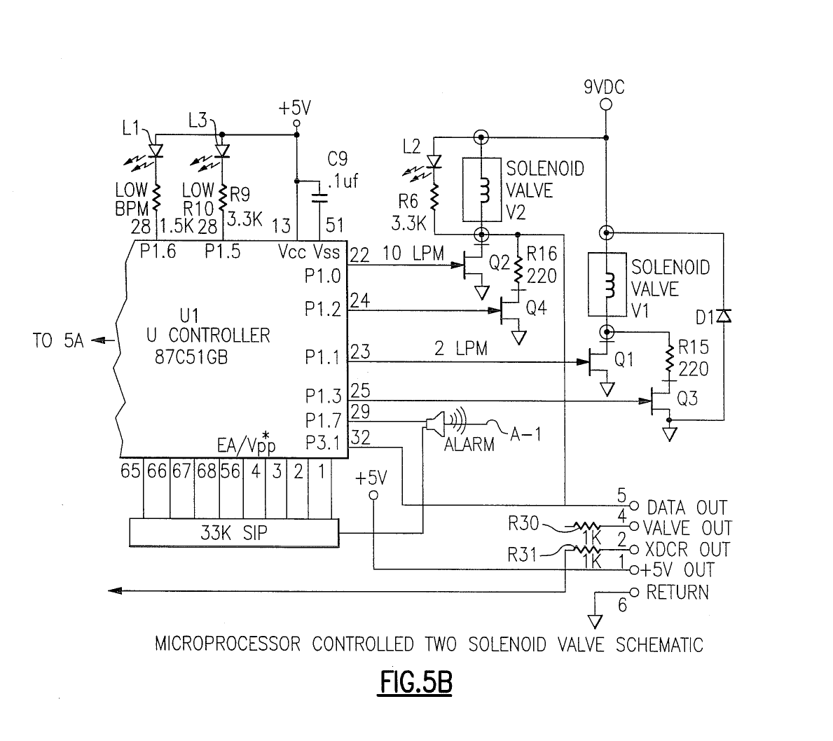

Again with reference to FIG. 2, the insufflation gas, in this case oxygen, is supplied from source 12. The oxygen is transmitted via gas supply tubing 40 to respective ones of first and second solenoid valves V1 and V2. Via lines 44 and 46, the gas communicates from first and second solenoid valves V1 and V2 with manifold 48. From manifold 48, gas is transmitted via gas delivery lube 52 to the nasal cannula 50. At least one sense tube 53 is also connected to the cannula 50, preferably isolated from communication with oxygen passing to the patient gas delivery tube 52. The sensing tube 53 is connected to sensor 32 which is a pressure transducer 32 supplied, for example, by SenSym Inc. of Palo Alto, Calif. The pressure transducer 32 is powered by power source 35 which uses power line 78 to supply either 110VAC converted to 5VDC by AC/DC, converter 80 or, alternatively, direct current from a battery 82 which is connected electrically in line with a battery low sensor 84 whose function will be more fully described hereinafter. Additional power outputs from the power source 35 are provided and designated PS. The PS power supply output is shown in FIG. 2 to communicate, via electrical power via lines 58 and 60, with first solenoid valve V1 and second solenoid valve V2. Power source 35 also provides electrical power to the microprocessor 34 via line 79. The output line 86 of the pressure transducer is also connected to an input in the microprocessor 34, which is also labeled U1 in FIGS. 5A and 5B.

In operation, the sensing tube 53 will be under positive pressure during a patient's exhalation and negative pressure during a patient's inhalation when the nasal cannula 50 is fitted to a normally breathing patient. Referring to FIG. 3, the top horizontal sinusoidal line represents a trace of a patient's breathing cycle where the curve above the straight horizontal line indicates the positive pressure in the sensing tube 53 (base line 20 in FIG. 2) during exhalation and the curve below the line represents the negative pressure in the sensing tube 53 during inhalation. The pressure differences over the period of a patient's breathing cycle are sensed by the pressure transducer which directly communicates with the pressure of the gas in the sensing tube 53. Typically, the pressure transducer will provide a proportional analog signal having positive and negative voltage values representative of the positive and negative pressure variants of a patient's exhalation and inhalation as shown by the graph on FIG. 3. This signal is fed via output line 86 to the microprocessor 34 or U1.

In the microprocessor 34 or U1, the positive and negative voltage containing signal stream or waveform is converted into a digital format and is continuously stored in the random access memory of the microprocessor U1. The stored digital signal is accessed continuously during the operation of the device for determination of the occurrence of various preselected conditions which actuate or trigger the operation of first and second solenoid valves V1 and V2. During the exhalation interval (see FIG. 3) of the immediate breathing cycle, the maximum positive pressure is indicated at positive peak pressure value 38. When the software in the microprocessor 34 or U1 verifies that a maximum value is reached, a predetermined fraction of that signal value is created by the microprocessor and the digitized, stored waveform signal is interrogated and compared with that created value. When that value is reached, an enable signal is produced in the microprocessor to activate valve driver 62 which, in turn, actuates first solenoid valve V1 opening it to the source 12 of oxygen via tubing 40 and the nasal cannula 50 via valve tubing 44 and 46 of respective ones of first and second solenoid valves V1 and V2 and gas delivery tube 52. The rate of flow of the oxygen is regulated by the size of an orifice (not shown) inherent in the valve and is typically about 2 liters per minute for first solenoid valve V1 although other sizes are possible.

Another preselected fraction of the maximum negative inhalation pressure is sensed. This value can be set by the respiratory therapist or patient to accommodate changes in physical activity and the set points will have been predetermined for each patient by monitoring blood gases during selected activities. Within the limits of adjustability of an amount of oxygen to be delivered, there can be incorporated in such a fixed flow device a degree of patient need accommodation not hitherto obtained.

Likewise, second solenoid valve V2 can be replaced with a variable orifice (not shown) or variable flow valve (not shown) which can be programmed to deliver the predetermined amount of oxygen insufflation gas during the inhalation interval before the predicted maximum negative pressure so as to take full advantage of the benefits and advantages of the present invention.

This oxygen continues to flow to the patient until the point in the breathing cycle when trigger point “LS” is reached. The trigger point is generated by the microprocessor 34 or U1 when the value of the pressure transducer output reaches a preselected fractional value of the peak value of the inhalation interval of the immediate breathing cycle, which was determined and stored by the microprocessor at the peak of the inhalation interval during the immediate breathing cycle. Contemporaneously, the second enable signal is routed to valve driver 62 which is energized/actuated causing oxygen to flow from gas supply tubing 40 and through valve tubing 46 from whence it exits through the gas delivery tube 52 to the patient. The rate of flow of oxygen is determined by the size of an orifice restrictor at the valve seat (not shown) of second solenoid valve V2. This oxygen flow continues until 33%, for example, of the peak negative pressure value of the inhalation interval of the immediate breathing cycle is reached in the subsequent inhalation interval. Simultaneously, the microprocessor 34 or U1 will be measuring the present inhalation interval to calculate and store the trigger point value, i.e., 33% of the negative peak pressure value of the immediate inhalation interval, for the generation of the next trigger point which is required during the succeeding breathing cycle.

The sequence described hereinbefore for the embodiments is repeated for each breathing cycle. If the patient's need for oxygen increases, e.g., from exertion or exercise, the appropriately programmed present invention automatically accommodates the increased need by delivering a predetermined amount of oxygen for each exhalation/inhalation interval of each breathing cycle. Operation of the present invention is further facilitated by the following switches, lights and an alarm. These are shown in FIGS. 5A and 5B:

(a) “TEST” Switch, TS-1, is a multi position digital switch which can be used by the operator to run a series of functional tests on the device to check its operation prior to placing the device into use with a patient. These tests can also be used as a diagnostic tool in the event of equipment malfunction.

(b) “LO BPM” is the label placed on light L-1, designating “Low Breaths Per Minute”. This light is illuminated by a signal from the microprocessor 34 or U1 when the patient's breathing rate decreases to an unsafe level.

(c) “ALARM” A-1 is sounded by a signal from the microprocessor 34 or U1 whenever the breathing rate is too low as determined in (b) above, or when the battery voltage decreases below a preset level which would provide for correct operation of the device. The present invention might include a switch so that when the alarm sounds, the patient could manually switch to the default rate of flow.

(d) “VALVE ON” light L-2 is a green light connected across either one or both of the solenoid valves, so that the light is illuminated whenever the valves or valve is activated thereby signaling the cycling of the valve(s) with each breath.

(e) “LO BAT” L-3 is the low battery light. This red light is illuminated by a signal from the microprocessor 34 or U1 at the same time that the ALARM is sounded. Additionally, this provides the information that the alarm is sounded, e.g., because the battery voltage was low and not that the patient was having breathing difficulty. Again, the patient may employ the switch for the default rate of flow when the low Battery light illuminates.

### DETAILED DESCRIPTION OF CIRCUITS

### Power Source

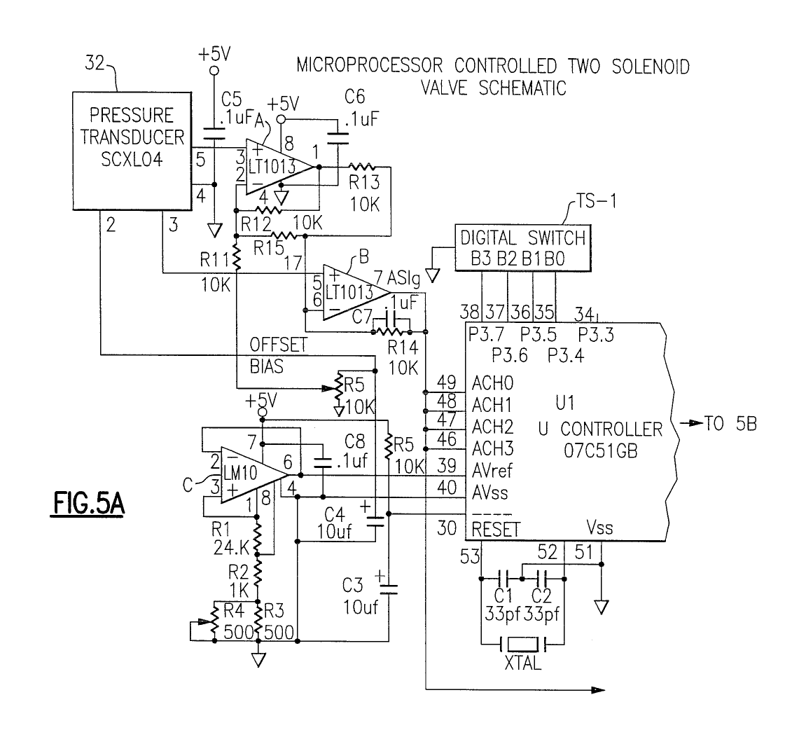

With reference to FIG. 4, the present invention is normally powered using 110VAC which is converted to 9VDC via the AC/DC converter 80. The 9VDC trickle charges the battery 82 through the charging resistor R17. The value of R17 is selected to prevent damage to the battery. When the AC/DC converter is unplugged from the system, the battery B1 provides backup power to the system. The diode D2 bypasses the charging resistor R17 to enable adequate system power in the backup mode. Capacitor C10 stores sufficient charge to supplement any large power demands when the solenoid valves are activated. The 9VDC is applied to the step-clown DC—DC converter circuit DC which provides .sup.+5VDC regulated power to the electronic circuits when switch SW1 is in the 0N position. The 9VDC is also applied to the solenoid valves V1 and V2. Converter DC is configured as a step-down converter. The resistor R19 is selected to limit the maximum output current at .sup.+5VDC. The filter circuit comprised of diode D3, inductor L4, and capacitor C11, smooths the output ripple to an acceptable level. Regulation is provided by feeding back the output signal to the SENSE input, pin 8, of the DC—DC converter. A low battery signal is generated in the DC—DC converter. The trip point is determined by the value of resistor R18, and the network of resistors R20, R21 and R22. The low battery signal AO is provided at pin 6 of the DC—DC converter and sent to the microprocessor input pin 34-P3.3, as shown in FIG. 5A.

Valve Driver/Power Saver

In FIG. 5B, the microprocessor 34 or U1 provides turn-on signals to actuate solenoid valves V1 and V2. The valves will remain actuated as long as the turn-on signal is present. The drive circuitry for the solenoid valve V1 consists of a MOS-FET semiconductor Q1 to actuate solenoid valve V1, and a MOS-FET Q3 with a resistor R15, to hold the valve in the actuated position at reduced power. The power saving feature operates by switching the turn-on signal from Q1 to Q3 immediately after the solenoid valve is actuated. The current required to hold the solenoid valve actuated is less than the current required for actuation and is set by selecting the value of R15. The diode D1 clamps the voltage across the solenoid valve to prevent arcing and overshoot. Similarly, the drive circuitry for the solenoid valve V2 consists of MOS-FETs Q2, Q4 and resistor R16. A light emitting diode L2, and its current limiting resistor R6, are placed across solenoid valve V2, to indicate that the valve has been actuated. Each turn-on signal will result in the illumination of the light emitting diode for the duration of the signal.

Alarm

In FIGS. 2 and 5B, the alarm is a piezoelectric device that emits an audible sound when activated by the microprocessor. The combinations of conditions to cause an alarm are programmed into the microprocessor. The alarm is sounded when any of the predetermined conditions are sensed.

Diagnostic Outputs

Signals are available to aid in data logging and troubleshooting. These signals can be accessed and displayed with the use of auxiliary equipment such as an oscilloscope, a chart recorder, etc.

Digital Switch

In FIGS. 2 and 5A, the digital switch TS-1 is a multi-position rotary switch that provides a four digit binary coded decimal (BCD) output. The output of the BCD switch is connected to the microprocessor at pins 35, 36, 37 and 38. The selected codes will address preprogrammed diagnostic routines that will perform calibration, system setup and diagnostic operations.

Reference Voltage Generator

In FIG. 5A, the reference voltage generator circuit consists of a reference voltage and operational amplifier C. Resistor R1 provides the feedback for the amplifier C. Resistors R2, R3 and POT R4 provide input resistance. POT R4 provides adjustability of the reference voltage output.

The precision reference voltage is utilized by the micro controller analog to digital converter for its reference voltage. Also, the reference voltage provides a precision and stable voltage to the pressure transducer bridge circuit.

The offset bias voltage utilized by the pressure transducer circuit is provided at the center tap of POT R5. The voltage is adjustable between 0 volts and the reference voltage.

Pressure Transducer Circuit

Also in FIG. 5A, the pressure transducer circuit consists of a standard differential pressure transducer 32 and differential amplifiers A and B. The pressure transducer is a typical variable resistance bridge circuit. The outputs of transducer 32 are connected to operational amplifiers A and B via output pins 5 and 3, respectively. Pin 2 is the reference voltage line and pin 4 is the return input (ground).

The operational amplifiers A and B are each configured as a differential amplifier with high gain. The offset bias voltage provides an offset output voltage at pin 7 of B also defined as ASIg. The output ASIg is adjusted for ½ the reference voltage at ambient pressure. The offset voltage provides a means to output positive and negative pressure measurements.

Micro Controller

The microprocessor U1 or 34, also known as micro controller, is a standard Intel Part MC80C51 GB, for example. The basic features are the following:

8 bit computer architecture;

256 random access memory;

4K programmable memory; and

8 channels of analog to digital conversion.

The crystal (XTAL), attached at pins 52 and 53, provides the control of the operating frequency for the micro controller. The reference voltage generator provides a power on reset signal (RESET) to the microprocessor. The signal is set to a low voltage upon initial power turn-on. The microprocessor is held inactive until the signal goes to a logic high level. At this time, the microprocessor starts executing stored programmed instructions. The process flow is discussed hereinafter. The input signals to the microprocessor are the analog pressure transducer signal Asig and digital signal Battery Lo (pin 34-P3.3). The Asig are inputted to the first four analog channels for digital conversion ACH0-pin 49, ACH1-pin 48, ACH2-pin 47, ACH3-pin 46.

Outputs from the microprocessor are the following digital signals (see FIG. 5B). P1.0-pin 22 and P1.1-pin 23. P1.0 commands the valve driver circuit V2 and P1.1 commands the valve driver circuit V1.

Output at P1.7-pin 29 is connected to an audible alarm (e.g., a buzzer) A-1. The microprocessor generates various audible frequencies to denote different alarm indications. The output P1.6-pin 28, P1.5-pin 27 drive light emitting diodes (LED) to indicate low breathing rate and Battery Lo voltage, respectively.

Program Flow

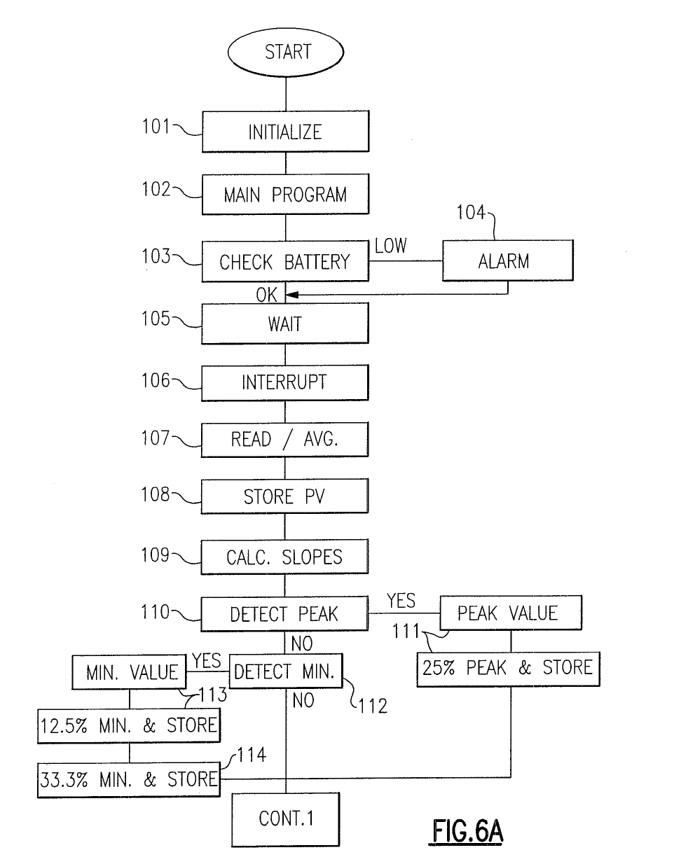

The following describes the process flow that will be coded into the micro controller. FIGS. 6A and 6B show the flow of the process that monitors the exhalation and inhalation pressures in real-time and processes this information to determine the start and stop time for turning on and off the O₂ valves.

Upon completion of the power-on reset, the stored program initializes the microprocessor. The initialization 101 consists of setting up the 10 ms interrupts timer, baud rate timer, serial port, A/D converter and input/output ports of the microprocessor. Once initialization has been completed, the program enters the main program 102. The main program starts with a check of the Battery Lo signal P3.3 at step 103. If the battery voltage level low is detected, the processor goes to the alarm routine at step 104. The processor turns on the Lo Battery LED indicator and also starts a low frequency beep on the audible alarm. Once completed, the program continues and goes into a wait mode at Step 105.

Upon receipt of a 10 ms interrupt, the program services the interrupt routine at step 106. This involves starting the analog-to-digital conversion and reset in the interrupt timer. The next step is to read the four analog converted voltages at 107 after a fixed time delay from the start of A/D conversion. This is to make sure conversion is complete. The four valves are averaged and labeled present value. The present value is stored at 108 into the last byte of the last in-first out (LIFO) memory of 16 bytes. Slopes are calculated 109 either as values or as indicators (positive or negative). Slopes are calculated between first and last, last and third to last, last and fifth to last, and third to last, and fifth to last. The flow continues by monitoring present value with the last highest value at step 110. If the present value is greater than the last highest value, the peak value is updated with the present value. If the present value is less than the peak value, the peak value is unchanged. To determine if a peak has been detected, the following conditions must be present:

1) the long slope must be negative (slope first to last);

2) the short slope must be negative (slope fifth to last and last);

3) in exhalation interval of the cycle.

If peak detection is enabled, the next step is to retrieve the peak value at step 111 and divide by 4 to get 25% of the peak value. This then becomes the start value for purge on.

The detection of the minimum value at 112 is very similar to the peak detection process with the following differences. Minimum detection criteria is the following:

1) the long-slope must be positive;

2) the short-slope must be positive;

3) in inhalation interval of the cycle.

When minimum detection has been detected, the minimum value is retrieved at 113, 114 and 12.5% and 33⅓% values are calculated and stored. These values are used for start of the main O₂ burst and turn-off on the next inhalation cycle. During the O₂ cycle in which the peak detection occurs, the 25% value of the peak is stored at step 115 and compared with the present pressure value. If the present value is less than or equal to the 25% value of peak, the microprocessor commands the valve driver V1 on at step 116. The V1 valve opens and provides a 2 L/M flow rate to purge the O₂ line.

During the same cycle and during the inhalation period, the processor compares the present pressure value with the 12.5% minimum value of the previous cycle at step 117. When the present pressure value equals or is less than the 12.5 minimum value, the microprocessor turns on the high flow valve V2 at step 118.

The valves are turned off when the present pressure value is greater or equal to 33⅓% of the minimum value of the previous cycle at steps 119, 120. The end of the main program flow, at step 121, shifts the LIFO memory by one byte for set up of the next 10 ms measurement.

Also, the watch dog timer is reset. The watch dog timer reinitializes the microprocessor if, for some reason, the program does not reset the watch dog timer.

The flow continues by monitoring the inhalation cycles. When no inhalation is detected, the microprocessor will turn on the audible alarm and LED indicator. Also, the Lo flow valve V1 will be enabled to provide continuous Lo O₂ flow.

The program flow continues with the process by returning to the start of main program and waiting for the 10 ms interrupt.

The intermittent gas-insufflation apparatus of the present invention provides significant advancements and benefits over the prior art. The intermittent gas-insufflation apparatus of the present invention determines the appropriate quantity of oxygen to be delivered to the patient during an immediate breathing cycle and adjusts appropriately to supply the quantity of oxygen commensurate with the physical activity of the patient.

The intermittent gas-insufflation apparatus delivers the appropriate quantity of oxygen continuously during an exhalation interval of the immediate breathing cycle and into an inhalation interval of a subsequent breathing cycle. This results in purging some of the air remaining in the nasal passage from the prior breath and enriches a remaining portion thereof. Further, a high-rate pulse of oxygen is delivered at approximately the beginning of the subsequent inhalation interval of the successive breathing cycle which is optimum. The intermittent gas-insufflation apparatus can determine an appropriate flow rate profile for delivering the oxygen during the exhalation interval of the immediate breathing cycle and the inhalation interval of the successive breathing cycle and, if desired, can modify the flow rate profile even while oxygen is being delivered to the patient. The intermittent gas-insufflation apparatus can terminate delivery of oxygen during the subsequent inhalation interval of the successive breathing cycle and, preferably before the negative peak pressure value generated in the immediate breathing cycle is reached in the successive breathing cycle. This feature conserves utilization of costly oxygen, particularly since oxygen is delivered when it could be best utilized by the patient.

As can be seen in FIGS. 2 and 5A, the system and method of the present invention relate to one or more transducers which detect or sense, due to pressure variations, the end of an exhalation breath and the beginning of an inhalation breath of a patient. The system is designed to predict the beginning of the next subsequent inhalation and activate delivery of the bolus during approximately the last 10% of the exhalation breath which is immediately prior to the next inhalation breath of the patient. Such deliver provides the system and method with a sufficiently amount of oxygen or room air, with an increased concentration of oxygen, along the oxygen supply tubing or conduit and to a patient interface, such a cannula with a pair of nares for positioning within the nostrils of the patient and delivery of the treatment gas or a divided cannula having a pair of nares in which a first one of the pair of nares communicates with the treatment gas to one of the nostrils and a second one of the pair of nares communicates with a sensing device for sensing pressure in the other nostril of the patient. The treatment gas is exhausted out through opening in the nares and enters into those nasal and/or mouth cavities and dilutes the concentration of carbon dioxide as well as enrich the concentration of oxygen of the exhaled gases contained in those cavities or areas so that when the patient commences the next inhalation breath, the gases initially inhaled by the patient contains a sufficiently diluted concentration of carbon dioxide as well as a sufficiently enriched concentration of oxygen.

Upon activation of the oxygen concentration system and method for a detected or sensed breathing cycle, the supply of oxygen, e.g., room air with a concentrated or increased level of oxygen, to the patient may continue for the entire remainder of the inhalation breath. In a preferred form of the system and method, the supply of oxygen may be discontinued prior to completion of the inhalation breath of the patient, e.g., during approximately the last 10% or so of the patient's inhalation breath, since the later portion of the supplied oxygen generally only actual reaches the nasal and/or mouth cavities or possibly the trachea at the time the patient's inhalation ceases and thus does not reach the lungs of the patient and is wasted. The discontinuance of the oxygen supply, prior to completion of inhalation, still results in both an adequate oxygen supply as well as conservation of oxygen.

Failsafe Modes of Operation

As discussed herein above, the conserver 6 of the present invention includes a method and apparatus for maintaining an acceptable level of treatment gas in the patient under fault conditions, such as a loss of inhalation/exhalation data from the patient or a loss of power in the system.

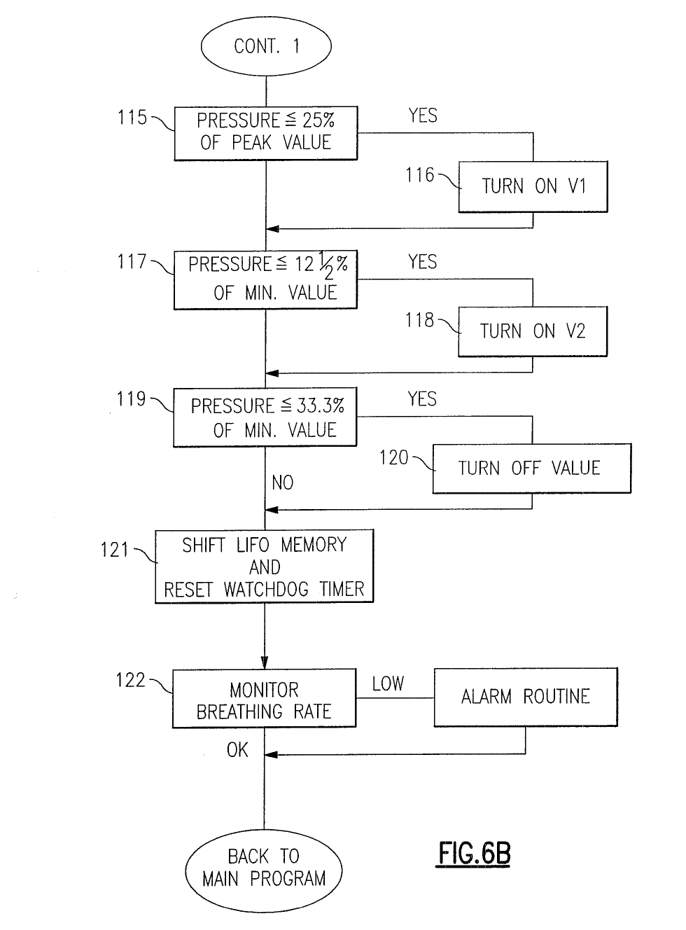

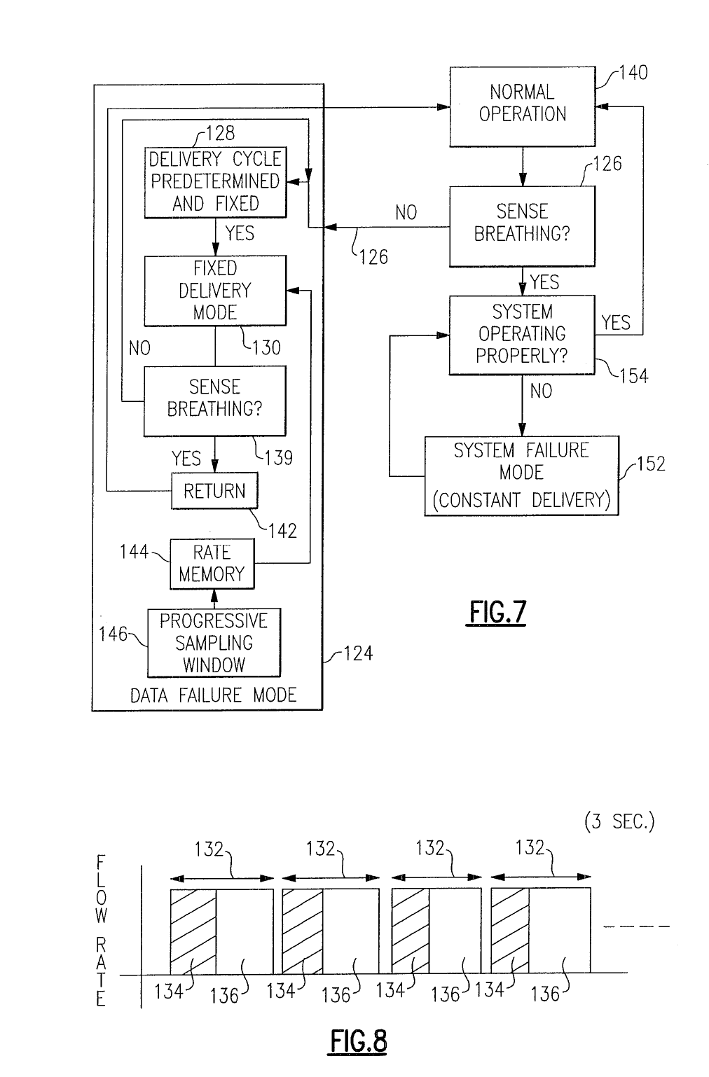

These methods and apparatus are illustrated, for example, in FIGS. 7 and 8, and are referred to as “failsafe” mechanisms or methods and operate to ensure an adequate level of treatment gas saturation in the patent. In a first failsafe mode, which may be referred to as a patient data failure mode, the system is required to maintain the desired level of treatment gas in the patient when the breathing cycle of the patient, that is, either an inhalation or exhalation breathing interval, can not be sensed or detected by the sensing device 32.

A second failsafe mode may be referred to as a system failure mode and may arise, for example, when there is a power failure in the apparatus, whether for internal or external reasons, or when at least some element of the system develops an operational fault or fails outright.

Again, the object of both the first and second failure modes of operation is to maintain an acceptable level of treatment gas in the patient under the fault conditions while also using as little power and oxygen as is necessary to maintain the desired level of treatment gas in the patient under the patient's current requirements, which will depend upon such factors as the patient's current condition, such as asleep or awake, and activity level.

Patient Data Failure Mode 124

First considering the patient data failure mode of operation as illustrated in FIG. 7, the conserver 6 enters patient data failure mode 124 when, in step 126, at least one sensing device 32, such as a transducer, a pneumatic diaphragm or some other sensing or detecting device 32, is unable to detect or sense the beginning or ending of an inhalation or exhalation breath of a patient 14. In patient data failure mode 124, the conserver 6 and method will assume that the patient 14 is still alive and breathing and will automatically switch to the data failure mode of failsafe operation to maintain an adequate level of treatment gas saturation in the patient under the patient's current condition and level of activity while conserving both the supply of treatment gas and power levels in the system.

In a first step 128 of the patient data failure mode 124 of operation, the conserver 6 will continue cycle the supply of oxygen to the patient in a periodic “on” and “off” cycle at a preselected number of cycles per minute wherein the selected cycle rate may be determined by the patient's previously established requirements, which may be adjusted from time to time, depending upon the patient's anticipated activity, such as sleeping, or may depend on such factors the patent's last known oxygen requirements. The patient's last known oxygen cycle rate and oxygen flow rate and oxygen concentration requirements will in turn typically depend, for example, upon the patient's last known breathing cycle rate and condition and level of activity, such as whether the patient 14 was sleeping or awake but at rest or was physically active.

First assuming that the patient's required cycle rate is preselected, as described briefly above, and as described above, the conserver 6 will enter the patient data failure mode 124 at step 126, at which point the patient 14 breathing cycle information will not be available to the conserver 6. The on/off cycle rate of the conserver 6 must therefore be selected to provide the desired level of treatment gas to the patient even when the period of the on/off cycle does not precisely match that of the patient's breathing cycle and even when the on/off cycle is not synchronized with the patient's inhalation and exhalation intervals. Stated another way, the period and duration of the on cycle of the conserver 6 must be selected such that the uncoordinated and unsynchronized overlap between the on interval of the conserver 6 cycle, that is, the periodic interval during which the treatment gas is effectively delivered to the patient 14, and the patient 14 actual breathing cycle interval during which the patient 14 effectively receives the treatment gas, must be sufficient to maintain the desired level of treatment gas in the patient 14. Again, the period and duration of the conserver 6 on interval will be dependent upon the state or activity of the patient, e.g., is the patient at rest, sleeping, walking, exercising, etc.

According to a present embodiment of the present invention, it has been determined that cycling the supply of oxygen or other treatment gas to the patient 14 “on” and “off” at a rate of between about 10 to 40 times per minute and typically about 20 times per minute will adequately saturate the patient with oxygen, e.g., maintain the patient at an oxygen saturation level of about 92% to 93%, while the patient is at rest or sleeping. This on/off cycle rate generally is not sufficient, however, when the patient is undergoing any significant activity. If the patient is undergoing activity, an increased number of “on” and “off” cycles per minute, e.g., 30-50 cycles per minute, may be required in order to obtain a blood oxygen saturation level of the patient at approximately 92% to 93%.

Assuming that the failsafe gas delivery cycle is predetermined and fixed, in step 128 the conserver 6 will enter a fixed delivery process 130 wherein the treatment gas, such as oxygen, is delivered to the patient 14 in a failure delivery cycle 132 (FIG. 8) having fixed intervals of gas delivery 134 and gas non-delivery 136. Assuming that the system is to operate during the patient data failure mode at a cycle rate of 20 cycles per minute, the system and method will then begin delivering oxygen to the patient in successive failure delivery cycles 132, each failure delivery cycle 132 having a period of approximately 3 seconds, which corresponds to a cyclic rate of 20 cycles per minute. As illustrated in FIG. 8, the oxygen will be delivered to the patient 14 during each 3 second failure delivery cycle 132, the treatment gas being provided to the patient 14 during a gas delivery interval 134 of about 1 second or so and then turned off for a subsequent gas non-delivery interval 136 of about 2 seconds or so. It has been found that in this mode of operation, the overlap between the periods when gas is delivered to the patient 14 and the periods when the patient 14 is, in fact, accepting the gas for a beneficial result sufficiently overlap to maintain the patient 14 at the desired levels of treatment gas saturation.

As indicated in FIG. 7, the conserver 6 will periodically attempt, in step 139, to determine whether the conserver 6 is still unable to detect or sense breathing of the patient, which may occur, for example, at the end of each 3 second failure delivery cycle 132 or during the non-delivery interval 136 of each failure delivery cycle 132 and, if no breathing cycle or inhalation or exhalation interval is detected, the conserver 6 will return to step 126 to continue in patient data failure mode 124. The failsafe mechanism will continuously repeat this routine until the system and method is either turned off or is finally able to detect or sense breathing of the patient.

In the event that the conserver 6 is turned “off” when the conserver is operating in the patient data failure mode 124, the conserver 6 will cease operating and the supply of oxygen will be completely discontinued. In the event that the conserver 6 is left in the “on” state and continues to operate in patient data failure mode 124 and patient breathing is again detected in step 139, the conserver 6 will return, at step 142, to normal operation 140 and the conserver 6 and will again automatically supply oxygen at the end of the exhalation intervals and prior to commencement of the inhalation intervals as described herein above.

As described herein above, the operation of conserver 6 and the patient data failure mode 124 method of operation of the present invention may be extended through additional steps and processes. For example, controller 34 of conserver 6 may include a rate memory 144 that determines and averages information regarding the patient 14 breathing cycle over a time progressive sampling window 146 that determines, for example, the average breathing cycle period and the average lengths of the inhalation and exhalation intervals within the sampling window 146 period. This information may then be used to determine the current requirements of the patient 14 when the conserver 6 enters the patient data failure mode 124 as, for example, percentages of the average breathing cycle period and the average lengths of the inhalation and exhalation intervals determines within the sampling window 146 period, which is terminated at the time conserver 6 detects the failure in patient data and enter patient data failure mode 124. According to this method of operation in patient data failure mode 124, therefore, the period of failure delivery cycle 132 and the lengths of gas delivery interval 134 and gas non-delivery interval 136 are not fixed, but are instead continuously adjusted or adapted according to the actual breathing requirements of the patient.

In yet another extension to the operation of conserver 6 and to the patient data failure mode 124 operation of the present invention, that is, in addition to either or both of the features of the above described patient data failure modes of operation, the conserver 6 may further include facilities for detecting a current activity level of the patient 14. For example, a conserver 6 of the present invention may include a connection detector or sensor 148 (FIG. 2) for determining whether the conserver 6 is connected into the system 10 or has been mechanically disconnected from the system 10, as when the patient 14 is moving about by means of extensions of gas delivery tube 52 and sensing tube 53 and is thereby possibly in an active state, or at least is not passively resting or asleep. Similar purposes may be met by the addition of a motion sensor 150 (FIG. 2) to conserver 6, which may then indicate whether the patient 14 is possibly in an active state or not, or is probably in a passive resting state or asleep, by detecting motion of the conserver 6, which would normally be in motion only when the patient 14 is physically active.

The response of the conserver 6 to the activity level of the patient 14 when the conserver 6 enters the patient data failure mode 124 is thereby more directly dependent upon the apparent activity level of the patient 14, as are the treatment gas requirements of the patient 14 for various patient activity levels. It will also be noted that the degree of reliability in interpreting the motion indications from, for example, the connection detector or sensor 148 or the motion sensor 150, will depend upon the means by which conserver 6 motion and this, hopefully, patient 14 activity, is detected. For example, the connection detector 148 is probably less reliability indicative of a patient's activity than the motion sensor 150 because a conserver 6 may operate remotely from the apparatus 10 for a number of reasons that do not reflect a higher level of activity, such as comfort while watching television or reading a book. In contrast, the motion sensor 150 of the conserver 6 detects and indicates actual movement of the conserver 6, which is usually the system element most closely associated with the patient 14 and which thereby more reliably indicates whether the conserver 6, and thus the patient 14, is actually in an active state. Also, no motion of the conserver 6 will be indicated unless there is actual motion of the conserver 6, thereby eliminating one more areas of uncertainty.

The action taken by conserver 6 as a result of the patient 14 activity level will, of course, depend upon the condition and problems of the patient 14 and, for example, on the needs of the patient 14 as a result of the range of activity normally engaged in by the patient 14. For example, the requirement of a patient 14 whose activities range from sleeping to sitting in a wheelchair will be much narrower, and the peak will be much lower, than those of a patient 14 having a more active life. In certain instances, therefore, the conserver 6 may respond to a measurement of probably patient activity level for a given patient 14 by delivering treatment gas to the patient only during every other delivery cycle 132, or possible every second or third delivery cycle, as this delivery rate may be sufficient to maintain the necessary levels of treatment gas in the patient's system. In other instances, however, the conserver 6 may respond to an activity measurement for a given patient 14 by delivering treatment gas to the patient 14 during each delivery cycle 132 in order to maintain the desired treatment gas levels in the patient 14.

In either instance, however, the conserver 6 may include consideration of the patient's apparent activity level in determining the level of treatment gas delivered to the patient 14.

System Failure Mode 152

As described above, conserver 6 may further support a second failsafe mode referred to as system failure mode 152, which may arise, for example, when there is a power failure in the apparatus, whether for internal or external reasons, or when at least some elements of the system develop operational faults or fail outright.

As illustrated in FIG. 7, conserver 6 will exit normal operation 140 and will enter system failure mode 152 at a step 154 when conserver 6 detects that the battery or other power supply device for the conserver 6 has failed or malfunctions or is inadequately low on power, or if such condition is pending, or if conserver 6 detects that another component of the conserver 6 has failed or malfunctions for some reason, or has a pending failure or malfunction. For example, the conserver 6 may detect that a line current power supply is not connected to a power line and that the reserve power stored in a battery power supply is decreasing at an abnormal rate or for an abnormal period or has fallen below a lower limit.

In step 154, a conserver 6 of the present invention will switch to system failure mode 152 in which conserver 6 operates in a continuous delivery mode to provide a continuous flow of treatment gas to the patient 14. In continuous delivery mode a valve is switched to an open state whereby the treatment gas, such as oxygen or room air or a mixture of gases, are continuously delivered to the patient 14 at a preset delivery rate, e.g., typically ranging between 1.5 and 6 liters per minute. As a result of this continuous supply of treatment gas, the conserver 6 no longer conserves the treatment gas delivered to the patient 14, such as oxygen or room air with an increased concentration of oxygen, but the patient is nevertheless still supplied with a sufficient amount of treatment gals so that the patient is, for example, adequately saturated with oxygen to achieve an oxygen blood saturation level of approximately 92% to 93%.

In this regard, it has been described herein above and illustrated in FIG. 2, for example, that a conserver 6 of the present invention includes a valve assembly 30 having a first solenoid valve V1 and a second solenoid valve V2 wherein first solenoid valve V1 is operative between a first closed state and a first opened state and second solenoid valve V2 is operative between a second closed state and a second opened state. Each of solenoid valves V1 and V2 is independently connected by gas supply tubing 40 in fluid communication to and between source 12 of the treatment gas and an entrance 16 into the respiratory system of patient 14. Respective ones of valve tubings 44 and 46 connect solenoid valves; V1 and V2 to manifold 48 which, in turn, is connected to a nasal cannula 50 via a single gas delivery tube 52. Solenoid valves V1 and V2 are independently connected electrically to controller 34 via line 54 and 56 and to power source 35 via lines 58 and 60 and have a valve driver 62 interposed in respective lines 54 and 56 wherein each valve driver 62 is electrically connected to power source 35 via respective lines 64 and 66. Each valve driver 62 is, in turn, electrically connected to controller 34 via lines 67 and 69.

As has also been described with reference to FIG. 2, solenoid valve V1 is operative to actuate from the first closed state to the first opened state during exhalation interval 22 of immediate breathing cycle 24 and from the first opened state to the first closed state at a later stage “LS” of subsequent inhalation interval 26 of successive breathing cycle 28. Thus, the treatment gas flows (solid line) as shown during exhalation interval 22 of immediate breathing cycle 24 which begins at a waning stage “WS” of exhalation interval of the immediate breathing cycle. Waning stage “WS” represents the first predetermined percentage multiplied by positive peak pressure value 38. When in the first opened state, the treatment gas flow builds to a steady state flow as shown by a flat solid line portion 68 of flow trace 70. Meanwhile, second solenoid valve V2 is operative to actuate from the second closed state to the second opened state at approximately a beginning stage “BS” of subsequent inhalation interval 26 of successive breathing cycle 28 thereby causing the enhanced opened state of valve assembly 30. Beginning stage “BS” represents the third predetermined percentage multiplied by the peak negative pressure value of the immediate breathing cycle which is used in the subsequent inhalation interval. In the second opened state of second solenoid valve V2, the additional treatment gas flows as a high flow-rate pulse reflected by the spiked solid line portion 72 of flow trace 70. The solenoid valve V2 is operative to actuate from the second opened state to the second closed state at later stage “LS” of subsequent inhalation interval 26 of successive breathing cycle 28. Later stage “LS” represents the second predetermined percentage multiplied by the negative peak pressure value of the immediate breathing cycle. Thus, although not by way of limitation, solenoid valve V1 and solenoid valve V2 actuate to their respective closed states simultaneously. Preferably, later stage “LS” occurs before the negative peak pressure value of the subsequent inhalation interval. Furthermore, solenoid valve V1 and solenoid valve V2 respectively actuate to the first closed state and the second closed state when the second predetermined percentage of negative peak pressure value 36 is achieved. In any event, treatment gas flows at a flow rate selected from a flow rate range of 0.5 liters per minute and 12 liters per minute inclusive.

In a presently preferred embodiment of a method for providing a continuous flow of treatment gas to the patient 14 in the continuous delivery mode is to employ a bypass valve V3 connected between source 12 of the treatment gas, such as pressurized liquid or gaseous oxygen or ambient air with an enriched oxygen level or some other gas, and gas delivery tube 52. Bypass valve V3 diverts flow of the treatment gas around the main flow control valve, such as solenoid valves V1 and V2 in FIG. 2, so that the treatment gas is supplied in a continuous flow via the bypass valve V3 and directly to entrance 16 into the respiratory system of patient 14, such as at nasal cannula 50.

When the conserver 6 is in normal operation 140, bypass valve V3 is in a first operative position which directs the treatment gas to solenoid valves V1 and V2 for intermittent supply to the patient 14, as necessary. In system failure mode 152, bypass valve V3 is energized by conserver 6 to a second operative position in which bypass valve V3 diverts the treatment gas around solenoid valves V1 and V2 and to a diversion conduit 156 which is connected to conduit 52 downstream from solenoid valves V1 and V2, thereby supplying a continuous flow of the treatment gas directly to patient. Bypass valve V3 may be implemented, for example, as a latching valve or some other conventional valve which has two stable positions and which will preferably automatically move to a stable open position upon a failure of power to bypass valve V3.

In an alternate embodiment, a single combined supply valve (not shown) can replace both solenoid valves V1 and V2 and the bypass valve V3. The combined supply valve must, however, have a normally “open” position that allows the flow of treatment gas therethrough to the facial interface and a biased “closed” position which interrupts or prevents the flow of treatment gas through the combined supply valve. That is, combined supply valve is not energized or powered during the “on” treatment gas supply cycle. That is, in the “on” part of the treatment gas supply cycle, combined supply valve is in its normally open position and unpowered state wherein the treatment gas is supplied to the patient. The combined supply valve is in the powered or energized state only during the “off” part of the treatment gas supply cycle whereby the combined supply valve is biased into its closed position where it interrupts or stops the flow of treatment gas to the patient.

Therefore, and because of the design and operation of combined supply valve, combined supply valve will automatically return to its normally “open” position and will thereby allow the continuous supply of oxygen to the patient at the pressure and flow rate determined by the regulator in the event of loss of power or a malfunction of some other component of the conserver 6.

Lastly in this regard, it must be noted that the flow of treatment gas to patient 14 when the conserver 6 is operation in the system failure mode 152 is controlled by a flow control regulator 158 located along the treatment gas delivery path and the treatment gas will be delivered to patient 14 at the pressure and the flow rate determined by the flow control regulator 158 when conserver 6 is in system failure mode 152. Depending upon the specific implementation of the apparatus 10, the patient 14 may, if necessary or desirable, adjust or alter the flow rate of the treatment gas by manipulation of the flow control regulator 158.

Therefore, if the flow control regulator 158 is, for example, set to deliver oxygen or some other treatment gas at a flow rate of 5 liters per minute and a pressure of 1 p.s.i., for example, the combined supply valve (not shown) will remain in its normally “open” position when conserver 6 is in the system failure mode 152 and will thereby not interrupt or prevent the continuous delivery of the treatment gas to the patient 14.

Since certain changes may be made in the above described improved conserver and method for conserving oxygen, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Wherefore, I/we claim:

1. A system for conserving supply of a treatment gas to a patient, the system comprising:
a source of the treatment gas;
a sensing device for sensing a breathing cycle of a patient;
a conserver for controlling intermittent supply of the treatment gas to the patient in response to the sensed breathing cycle;
wherein the conserver operates in a first mode when the sensing device senses the breathing cycle to supply the treatment gas to the patient in a first intermittent cycle coordinated with the breathing cycle; and
the conservator operates in a second mode, when the sensing device is unable to sense the breathing cycle, in which the conserver supplies the treatment gas to the patient at a second intermittent cycle determined independently of the patient breathing cycle where the second intermittent cycle is selected to overlap an assumed patient breathing cycle such that at least a desired amount of the treatment gas is supplied to the patient.

2. The system according to claim 1, wherein during operation of the system in the second mode, the system has a treatment gas delivery period that is less than a duration of an interruption period.

3. The system according to claim 1, wherein during operation of the system in the second mode, the system delivers treatment gas to the patient at a rate ranging from ten to forty gas delivery periods per minute.

4. The system according to claim 3, wherein during operation of the system in the second mode, the system delivers treatment gas to the patient at a rate of twenty treatment gas delivery periods per minute.

5. The system according to claim 1, wherein during operation of the system in the second mode, the system delivers treatment gas to the patient a rate sufficient to maintain saturation of the patient.

6. The system according to claim 1, wherein the source of a treatment gas is one of a liquid oxygen source, a gaseous oxygen source and a concentrator for removing nitrogen from air and increasing a concentration of oxygen of the room air supplied as the treatment gas.

7. The system according to claim 1, wherein the conserver is coupled to a patient respiratory system interface which supplies the treatment gas to the patient.

8. The system according to claim 7, wherein the patient interface is a cannula with a first and second nares located for positioning within the nostrils of the patient for delivery of the treatment gas and for sensing a breathing cycle of the patient.

9. The system according to claim 8, wherein the cannula is a divided cannula and the first nare communicates with the conserver for delivering the treatment gas to one of the nostrils of the patient and the second nare communicates with the sensing device for sensing a pressure in the other nostril of the patient.

10. The system according to claim 1, wherein the sensing device is one of a transducer and a pneumatic diaphragm.

11. The system according to claim 1, wherein the conserver includes a concentrator for removing nitrogen from air and increasing a concentration of oxygen of the room air supplied as the treatment gas.

12. The system according to claim 3, wherein the conserver includes a patient activity sensing device for indicating an activity level of the patient and the patient activity sensing device facilitates adjustment of a duration of the treatment gas delivery period to the patient according to a sensed activity level of the patient.

13. The system according to claim 12, wherein the conserver, in response to a predetermined sensed activity level of the patient, adjusts the treatment gas delivery period such that the treatment gas is delivered to the patient only during every other treatment gas delivery period.

14. The system according to claim 1, wherein the conserver, in response to at least one of a power supply failure and a system component failure, operates a bypass valve connected between the treatment gas source and the patient interface to enter the open state to allow a continuous flow of the treatment gas to the patient interface.

15. The system according to claim 14, wherein the conserver further includes a regulator connected in line with the gas source and the bypass valve to control at least one of a flow rate and a pressure of the treatment gas to the patient interface.

16. The system according to claim 14, wherein upon cessation of power to the bypass valve, the bypass valve automatically enters an open state.

17. A system for maintaining a predetermined level of a treatment gas in a patient while conserving use of the treatment gas, the system comprising:
a source of oxygen;
a sensing device for sensing a breathing cycle of a patient;
a conserver for controlling intermittent supply of the oxygen to the patient in response to the sensed breathing cycle;
wherein the conserver operates in a first mode when the sensing device senses the breathing cycle to supply the oxygen to the patient in a first intermittent cycle coordinated with the breathing cycle immediately prior to the patient commencing inhalation so that the supplied oxygen dilutes and diffuses any carbon dioxide contained in the exhaled breath of the patient while also enriching a concentration of oxygen available for inhalation by the patient during a next inhalation breath;
the conservator operates in a second mode, when the sensing device is unable to sense the breathing cycle, in which the conserver supplies the oxygen to the patient at a second intermittent cycle determined independently of the patient breathing cycle;
the second intermittent cycle is selected to overlap an assumed patient breathing cycle such that at least a desired amount of the treatment gas is supplied to the patient; and
the conserver, in responsive to at least one of a power supply failure and a system component failure, is actuated to an open state to allow a continuous flow of the oxygen to the patient interface.

18. The system according to claim 17, wherein the conserver includes a patient activity sensing device for sending an activity level of the patient and the patient activity sensing device facilitates adjustment of a duration of an oxygen delivery period to the patient according to the sensed activity level of the patient; and the conserver, in response to a predetermined sensed activity level of the patient, adjusts the oxygen delivery period such that the oxygen is delivered to the patient only during every other oxygen delivery period.

19. A method for maintaining a predetermined level of a treatment gas in a patient while conserving use of the treatment gas by delivering the treatment gas from a treatment gas source and to a patient interface via a conserver connected between the treatment gas source and the patient interface, the method comprising the steps of:
sensing parameters of a breathing cycle of the patient;
controlling operation of the conserver according to the sensed parameters of the breathing cycle so that the conserver operates in one of a first mode and a second mode;
during the first mode, when at least one parameter of the breathing cycle is sensed, the conserver supplying the treatment gas to the patient in a first intermittent cycle coordinated with the patient breathing cycle, and
during the second mode, when the conserver is unable to sense at least one parameter of the breathing cycle, the conserver supplying the treatment gas to the patient in a second intermittent cycle determined independently of the patient breathing cycle, in which the second intermittent cycle is selected to overlap an assumed patient breathing cycle such that at least a desired level of the treatment gas is maintained in the patient.

20. The method according to claim 19, further comprising the step of, during the second mode of operation, using a treatment gas delivery period that is approximately one half a duration of a gas interruption period.

21. The method according to claim 19, further comprising the step of, during the second mode of operation, delivering the treatment gas at a rate ranging from ten to forty treatment gas delivery periods per minute.

22. The method according to claim 21, further comprising the step of, during the second mode of operation, delivering the treatment gas at a rate of approximately twenty treatment gas delivery periods per minute.

23. The method according to claim 21, further comprising the step of, during the second mode of operation, delivering the treatment gas at a rate sufficient to maintain saturation of the patient with the treatment gas.

24. The method according to claim 19, further comprising the step of using one of a liquid oxygen source, a gaseous oxygen source and a concentrator as the source of the treatment gas.

25. The method according to claim 19, further comprising the step of using a divided cannula and communicating a first nare with the conserver for delivering the treatment gas to one of the nostrils of the patient and communicating a second nare with the sensing device for sensing pressure in the other nostril of the patient.

26. The method according to claim 19, further comprising the step of using one of a transducer and a pneumatic diaphragm as the sensing device.

27. The method according to claim 19, further comprising the step of providing the conserver with a concentrator for removing nitrogen from air and increasing a concentration of oxygen to be supplied as the treatment gas.

28. The method according to claim 19, further comprising the step of providing the conserver with a patient activity sensing device for sensing an activity level of the patient and the patient activity sensing device facilitates adjustment of a duration of the treatment gas delivery period to the patient according to a sensed activity level of the patient.

29. The method according to claim 28, further comprising the step of the conserver, in response to a predetermined sensed activity level of the patient, adjusting the treatment gas delivery period such that the treatment gas is delivered to the patient only during every other treatment gas delivery period.

30. The method according to claim 19, further including the steps of:
detecting one of a power supply failure and a system component failure, and
actuating a bypass valve, connected between the treatment gas source and the patient interface, to an open state and allow a continuous flow of the treatment gas to the patient interface.

31. The method according to claim 30 further including the step of determining the flow of treatment gas to the patient interface by a regulator connected in line with the gas source and the bypass valve.

32. The method according to claim 30 further including the step of the bypass valve automatically being actuated to the open state upon cessation of power to the bypass valve.

33. A system for conserving supply of a treatment gas to a patient, the system comprising:
a source of the treatment gas;
a sensing device for sensing a breathing cycle of a patient;
a conserver for controlling intermittent supply of the treatment gas to the patient in response to the sensed breathing cycle;
wherein the conserver supplies the treatment gas to the patient at an intermittent cycle determined independently of the patient breathing cycle where the intermittent cycle is selected to overlap an assumed patient breathing cycle such that at least a desired amount of the treatment gas is supplied to the patient.
